(19) United States
(12) Patent Application Publication
(10) Pub. No.: US 2008/0234391 A1
(43) Pub. Date: Sep. 25, 2008

(54) Synthesis of Reversible Shell Crosslinked Nanostructures

(76) Inventors: Charles L. McCormick, Hattiesburg, MS (US); Brad S. Lokitz, Knoxville, TN (US); Yuting Li, Sheffield (GB)

Correspondence Address: HOWREY LLP-HN, C/O IP DOCKETING DEPARTMENT, 2941 FAIRVIEW PARK DRIVE, SUITE 200, FALLS CHURCH, VA, 22042-7195, US

(21) Appl. No.: 12/048,680

(22) Filed: Mar. 14, 2008

Related U.S. Application Data
(60) Provisional application No. 60/919,294, filed on Mar. 21, 2007.
(60) Provisional application No. 60/919,249, filed on Mar. 21, 2007.

(51) Int. Cl. A61K 47/32; C08L 53/00

(52) U.S. Cl. 514/772.1; 525/88

(57) ABSTRACT

In one aspect, the present invention is directed to a thermally responsive AB diblock copolymer prepared by RAFT polymerization wherein the diblock copolymer comprises poly(N-(3-aminopropyl)methacrylamide hydrochloride)-block-(N-isopropylacrylamide). Nanostructures of the thermally responsive diblock copolymer are formed by molecularly dissolving the diblock copolymer in aqueous solution at room temperature; and increasing the solution temperature to form nanostructures, for example vesicles or micelles. The first RAFT polymerization of an unprotected amino acid based monomer directly in water is also disclosed. The present invention also provides a method of forming shell cross-linked vesicles by adding a RAFT synthesized anionic homopolymer to a solution of the thermally responsive diblock copolymer. A method of forming interpolyelectrolyte complexed micelles or vesicles is also disclosed, the method comprising preparing by sequential aqueous RAFT polymerization a block copolymer comprised of N,N,-dimethyl acrylamide (DMA), N-acryloyl alanine (AAL) and N-isopropyl acrylamide (NIPAM); dissolving the block copolymers into aqueous solution; raising the solution temperature above the lower critical solution temperature of the NIPAM block; allowing the micelle solution to equilibrate; adjusting the pH of the solution to about 5; adding a cationic polymer to the solution; and stirring the solution. The reaction is readily reversed by the addition of a salt solution. In another aspect of the invention a reversible shell cross-linked micelle of a triblock copolymer cross-linked with cystamine is disclosed where a cleaving agent can be added to cleave the micelles. The reaction can be reversed with the addition of tris(2-carboxyethyl)phosphine or dithiothreitol.

This application claims benefit of priority to U.S. provisional application Ser. No. 60/919,294 filed Mar. 21, 2007, the entire contents of which are incorporated by reference herein.

The United States government may own rights to this invention pursuant to grants provided by the Department of Energy (DE-FC26-01BC15317) and the MRSEC program of the National Science Foundation (DR-0213883)

### BACKGROUND OF THE INVENTION

The ability of well-defined amphiphilic block copolymers to self-assemble into nanostructures such as micelles has attracted a great deal of interest for potential applications in the targeted delivery and controlled release of active agents. (15-27) Self-assembly from unimers to micelles is typically triggered by an external stimulus such as pH, temperature, or added electrolyte, and must occur above the critical micelle concentration (CMC) of the block copolymer. Applications in which the micelles undergo dilution can cause the polymer concentration to fall below the CMC and lead to the dissociation of the micelles into unimers. To circumvent this apparent limitation, several groups are currently exploring the covalent stabilization of micelles through chemical cross-links. These covalently stabilized micelles are commonly referred to as shell cross-linked (SCL) micelles and were first reported by Wooley and co-workers in 1996. (28) Several shell cross-linking methods have been reported in the literature including carbodiimide coupling of carboxylic acid groups via diamines, (29, 30) quaternization of amino groups, (28, 31-33) the reaction of hydroxyl groups with divinyl sulfone, (34) and UV-induced coupling of cinnamoyl groups. (35)

Traditional cross-linking technologies in pharmaceutical and other controlled delivery applications are often limited by a number of factors including poor reagent solubility and low reaction efficiency. Additionally, these reagents are often toxic and must be removed prior to use, (16, 27, 30, 35) One approach involves the complexation of charged segments incorporated into the micelle with oppositely charged polymeric cross-linkers to form polyelectrolyte complexed micelles. (13) Polyelectrolyte complexation is reported to have advantages over traditional cross-linking reactions in that products exhibit low toxicity and physical cross-links are formed without altering the activity of the reactive agent. Additionally, polyelectrolyte complexation produces no by-products and is reversible in the presence of added salt. (13)

N-Acryloyl derivatives of amino acids can be synthesized in a facile manner by reaction with acryloyl chloride. These functional biomimetic monomers are of interest due to their amphoteric nature, chirality, and ability to self-assemble into higher order structures. (36-39) Rapid developments of controlled radical polymerization techniques now allow the preparation of well-defined functional polymers and block copolymers.(40-42) More specifically, reversible addition fragmentation chain transfer (RAFT) (9, 42-44) polymerization allows for the direct synthesis of several acrylamido and methacrylamido-monomers and the formation of complex architectures including well-defined stimuli-responsive block copolymers. For example, our group has reported the aqueous RAFT polymerization and copolymerization of hydrophilic (meth)acrylamido monomers including anionic, (9, 45, 46) cationic, (9,47) zwitterionic, (9, 48, 49) and neutral derivitives. (9, 14, 50-54) There exists a need for additional RAFT polymerization techniques in aqueous solutions. A distinct advantage would be the ability to polymerize an unprotected amino-acid-based monomer, long useful in the polymer arts, directly in water.

Vesicles composed of lipid molecules play an important role in several biological functions including the storage and transportation of small molecules. Vesicles formed from the self-assembly of amphiphilic block copolymers are often more durable than conventional liposomes and have recently been the focus of research. Among them, chemically shell cross-linked vesicles reported by Du and Chen et al. are quite stable under extreme conditions.⁵ Typical methods of vesicle formation from amphiphilic block copolymers involve the use of organic solvent such as THF, DMF or dioxane, and require further purification processes which can be time-consuming and problematic. In addition, the self assembly process is highly dependent on the rate of dialysis or the addition of another solvent which is difficult to control. Recently, stimuli-responsive (pH sensitive) block copolymers that self-assemble into vesicles without the addition of organic solvents have been reported.

Drawbacks of using previously reported SCL micelles in drug delivery applications are that the crosslinks are non-degradable and the large sizes of the resulting structures preclude renal excretion. (66) It would be an advancement in the art to provide for the formation of SCL micelles that would possess reversible crosslinks, allowing nanostructure dissociation and renal excretion of the constituents. Because of the stringent pH requirements of the mammalian system, a block copolymer system that self-assembled in water without the need for varying the pH, for example, a thermally responsive polymer block copolymer system, would be an improvement in the art. Additionally, new methods of reversible cross-linking would advance this art.

### SUMMARY OF THE INVENTION

A method of forming interpolyelectrolyte complexed micelles is disclosed, the method comprising preparing by sequential aqueous RALFT polymerization a block copolymer comprised of N,N,-dimethyl acrylamide (DMA), N-acryloyl alanine (AAL) or N-acryloyl valine (AVAL) and N-isopropyl acrylamide (NIPAM); dissolving the block copolymers into aqueous solution; raising the solution temperature above the lower critical solution temperature of the NIPAM block; allowing the micelle solution to equilibrate; adjusting the pH of the solution to above 5; adding a cationic polymer to the solution; and stirring the solution. The reaction is readily reversed by the addition of a salt solution. Block copolymers of N,N,-dimethyl acrylamide (DMA), N-acryloyl alanine (AAL) or N-acryloyl valine (AVAL) and N-isopropyl acrylamide (NIPAM) prepared via sequential aqueous RAFT polymerization are shown to undergo a reversible, temperature-induced unimer-to-micelle transition. Above the phase transition temperature, the resulting micelles are cross-linked via interpolyelectrolyte complexation of the poly(AAL) or poly(AVAL) segments in the hydrated shell with a cationic homopolymer. These interpolyelectrolyte complexed micelles remain intact upon cooling below the lower critical solution temperature. In one aspect of the invention, the reversibility of the crosslinking and associated loss of nanostructure integrity occurs with the addition of simple electrolytes. To our knowledge, this represents not only a unique method of producing thermally responsive reversible cross-linked nanostructures but also the first RAFT polymerization of an unprotected amino acid based monomer directly in water.

In one aspect, the present invention is directed to a thermally responsive AB diblock copolymer prepared by RAFT polymerization wherein the diblock copolymer comprises poly(N-(3-aminopropyl)methacrylamide hydrochloride)-block-(N-isopropylacrylamide). Aggregates of the thermally responsive AB diblock copolymer are formed by molecularly dissolving the diblock copolymer in aqueous solution at room temperature; and increasing the solution temperature to form aggregates. Herein we disclose the first example of vesicle formation from the self-assembly of hydrophilic-hydrophilic block copolymers directly in water by variation of the solution temperature. The resultant vesicles are shell crosslinked through polyelectrolyte complexation. Thermally responsive AB diblock copolymers were successfully prepared by RAFT polymerization. At room temperature, these block copolymers exist as unimers in aqueous solution and self-assemble into vesicles when the solution temperature is increased. Both solution concentration and heating rate influence the size and size distribution of the vesicles. These vesicles can be successfully cross-linked (structure “locked”) by adding an oppositely charged polyelectrolyte.

SCL micelles formed with cystamine, a reversible cross-linking agent are disclosed herein. An important advantage of such micelles is that, in principle, the block copolymer chain should be readily eliminated from the body after in vivo micelle degradation. In one aspect of the invention the individual copolymer chains produced after cleavage of the disulfide bonds in the cystamine linkages can be re-assembled into micelles and subsequently re-crosslinked.

The present invention also provides method of forming shell cross-linked vesicles by adding a RAFT synthesized anionic homopolymer to a solution of the thermally responsive AB diblock copolymer. In another aspect of the invention a reversible shell cross-linked micelle of a ABC triblock copolymer cross-linked with cystamine is disclosed where a cleaving agent can be added to cleave the micelles. The reaction can be reversed with the addition of cystamine. In one embodiment the thermally responsive micelles are based on a thermo-responsive ABC triblock co-polymer. The copolymer is shell crosslinked. The micelles prepared from a novel thermo-responsive ABC triblock copolymer self-assembles in aqueous solution. In one aspect, the SCL micelles are cleaved using different types of cleaving agents. In one embodiment, the cleaving process is fully reversible. In another aspect the micelles that are cleaved can be re-formed using cystamine as a thiol-exchange compound. In another aspect, the SCL micelles can be used as potential nanoscale drug delivery carriers wherein the release rate can be easily controlled. In summary, triblock copolymer micelles were prepared from a novel thermo-responsive ABC triblock copolymer that self-assembles in aqueous solution.

The present invention provides a thermally responsive, reversible crosslinked nanostructure comprising a multiblock copolymer comprising a first block of a charged polymer, and a second block of a thermally responsive polymer, said multiblock copolymer crosslinked with a polymer having the opposite charge of the first block polymer. The nanostructure is a vesicle or micelle. In a preferred embodiment the nanostructure further comprises a bioactive compound. Exemplary bioactive agents include antibiotics, antiviral drugs, anticoagulants, non-steroidal anti-inflammatory drugs (NSAIDs), steroidal anti-inflammatory drugs, hormones, chemotherapeutic agents, antimetabolites, alkaloids, enzymes, and taxanes.

In accordance with the present invention the thermally responsive polymer is a polymer that becomes hydrophobic and associates into nanostructures such as micelles and vesicles in water above a temperature known as the lower critical solution temperature (LCST). Such polymers are hydrophilic below the LCST and therefore undergo reversible temperature induced unimer to nanostructure transition. For instance, poly(N-isopropylacrylamide) (NIPAM) has an LCST of 32° C. A most preferred thermally responsive copolymer is poly(N-isopropylacrylamide) (NIPAM). Other examples include poly(2-[(N-morpholino)ethyl]methacrylate), poly(N-acryloylpyrrolidine), poly(N-acryloylpiperidine), and poly(N-n-propylacrylamide).

In the nanostructure of the present invention the charged polymer is an anionic or cationic polymer. Examples of anionic polymers include sulfonated or carboxylated polystyrene, sulfonated or carboxylated polyacrylamide, sulfonated or carboxylated polymethacrylamide, sulfonated or carboxylated polyacrylate, sulfonated or carboxylated polymethacrylate or poly(acryloyl amino acids). Exemplary examples of anionic polymer are poly(sodium 2-acrylamido-2-methylpropanesulfonate) (PAMPS), poly(N-acryloyl alanine) (AAL) or poly(N-acryloyl valine) (AVAL). Examples of cationic polymers include polyvinylbenzylamine, polyaminomethacrylamide, polyaminomethacylate, or polyvinylpyrridines. Exemplary cationic polymers are poly[(ar-vinyl benzyl) ammonium chloride] (PVBTAC) or poly(N-(3-aminopropyl)methacrylamide hydrochloride) (AMPA).

In another aspect of the invention, the nanostructure further comprises a third block of a nonionic, hydrophilic polymer. The nonionic, hydrophilic polymer can be a polyacrylamide, polymethacrylamide, polyacryloyl morpholine, polyvinyl pyrrolidone, polyacryloyl pyrrolide, glycopolymer, polyethylene glycol methacrylate, polyalkylene oxide, polyvinyl alcohol. A preferred nonionic, hydrophilic polymer is poly(N,N,-dimethyl acrylamide) (DMA) or block-poly(ethylene oxide) (PEO). A preferred arrangement of the triblock copolymer comprises block [nonionic, hydrophilic polymer]-block [charged polymer]-block [thermally responsive polymer], e.g. poly(DMA)-block-poly(AAL)-block-poly(NIPAM) or poly(DMA)-block-poly(AVAL)-block-poly(NIPAM).

In yet another aspect of the invention, there is provided a thermally responsive, reversible crosslinked nanostructure composed of a multiblock copolymer comprising a first block of a statistical copolymer of N-acryloxysuccinimide (NAS) or N-methacryloxysuccinimide (NMS) and a hydrophilic monomer, and a second block of a thermally responsive polymer, said multiblock copolymer crosslinked with cystamine. The nanostructure is a vesicle or micelle. In a preferred embodiment the nanostructure is further comprised of a bioactive compound. In accordance with the present invention the thermally responsive polymer, such as poly(N-isopropylacrylamide) (NIPAM). In a preferred embodiment the first block is poly[(DMA)-stat-(NAS)]. Further the multiblock copolymer may include a third block of a nonionic, hydrophilic polymer. Examples of nonionic, hydrophilic polymers include polyacrylamide, polymethacrylamide, polyacryloyl morpholine, polyvinyl pyrrolidone, polyacryloyl pyrrolide, glycopolymer, polyethylene glycol methacrylate, polyalkylene oxide, polyvinyl alcohol. A preferred example of the third block is poly(DMA) or (PEO). A preferred example of the multiblock copolymer is (PEO)-block-poly[(DMA)-stat-(NAS)]-block-poly(NIPAM).

### BRIEF DESCRIPTION OF DRAWINGS

FIG. 1 depicts the variation of hydrodynamic diameter with temperature for a 0.5% (w/w) aqueous solution of the diblock copolymers (heat rate=0.1° C./min).

FIG. 2 is the Dynamic light scattering size distribution of the PAMPA₈₈-PNIPAM₅₀ diblock copolymer under specific conditions: a) 0.05% aqueous solution at 45° C.; b) 0.05% aqueous solution with PAMPS, 45° C.; c) 0.05% aqueous solution with PAMPS, 25° C.

FIG. 3 is the ¹H NMR spectra of the homo and block copolymers at selected temperatures.

FIG. 4 depicts the pseudo first order kinetic plot for the 4-Cyanopentanoic acid dithiobenzoate (CTP) and 2-ethylsulfanylthiocarbonylsulfonyl-2-methyl-propionic acid (EMP) mediated homopolymerizations of N-acryloyl alanine (AAL) at 70 C.

FIG. 5 depicts the number average molecular weight (Mₙ) and PDI (M_{w}/Mₙ) versus conversion for the aqueous homopolymerizations of N-acryloyl alanine (AAL) mediated by -Cyanopentanoic acid dithiobenzoate (CTP) and 2-ethylsulfanylthiocarbonylsulfonyl-2-methyl-propionic acid (EMP) at 70° C.

FIG. 6 is a proton nuclear magnetic resonance (¹H NMR) spectra for block copolymers of N,N-dimethylacrylamide (DMA), N-acryloyl alanine (AAL), and N-isopropyl acrylamide (NIPAM): DMA₁₀₀-b-AAL₆₅-b-NIPAM₁₆₅ (i), DMA₁₀₀-b-AAL₆₅ (ii), and DMA₁₀₀ (iii).

FIG. 7 depicts (A) Refractive index (RI) traces and (B) cumulative weight fractions for the chain extension of the N,N-dimethylacrylamide (DMA) macro-chain transfer agent (macroCTA) (-DMA macroCTA, number average molecular weight (Mₙ)=9 900 g/mol; polydispersity (M_{w}/Mₙ=1.07) with N-acryloyl alanine (AAL) (-DMA₁₀₀-b-AAL₆₅, Mₙ=20 100 g/mol; M_{w}/Mₙ=1.13) showing the evolution of molar mass with time. (C) RI traces for the ABC triblock copolymer of DMA, AAL, and N-isopropyl acrylamide (NIPAM) (-DMA₁₀₀-b-AAL₆₅-b-NIPAM164 Mₙ=28 400 g/mol; M_{w}/Mₙ=1.18) and the ABCBA pentablock copolymer (-DMA₅₃-b-AAL₃₈-b-NIPAM₃₄₅-b-AAL₃₈-b-DMA₅₃, Mₙ=60 400 g/mol; PDI=1.16).

FIG. 8 depicts apparent hydrodynamic diameters (Dₕ) for the block copolymers of N,N-dimethylacrylamide (DMA), N-acryloyl alanine (AAL), and N-isopropyl acrylamide (NIPAM) measured by dynamic light scattering (polymer concentration=1.0 g/L) as a function of Temperature.

FIG. 9 depicts apparent hydrodynamic diameters (Dₕ) for the ionically cross-linked micelles and copolymer comprised of N,N-dimethylacrylamide (DMA), N-acryloyl alanine (AAL), and N-isopropyl acrylamide (NIPAM) (DMA₁₀₀-b-AAL₆₅-b-NIPAM1₁₆₅, polymer concentration=1.0 g/L) as a function of sodium chloride concentration ([NaCl]).

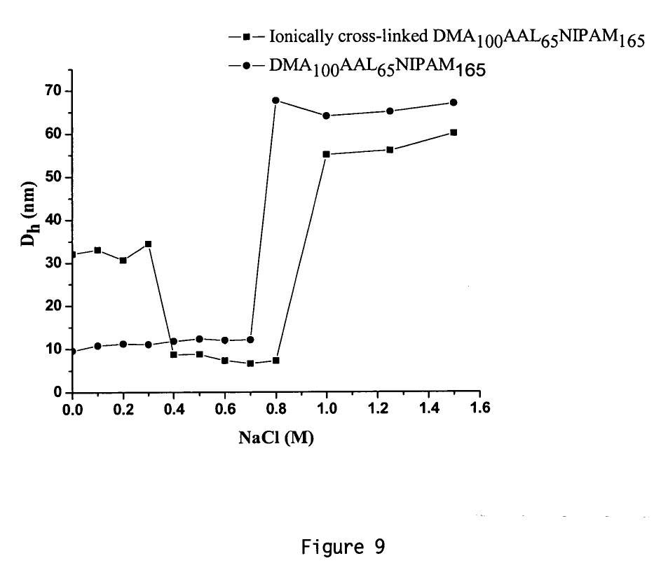

FIG. 10 is ¹H NMR spectra (2% w/w in D₂O) for copolymers comprised of N,N-dimethylacrylamide (DMA), N-acryloyl alanine (AAL), and N-isopropyl acrylamide (NIPAM)-DMA₁₀₀-b-AAL₆₅-b-NIPAM₁₆₅ unimers at 25° C. (i), DMA₁₀₀-b-AAL₆₅-b-NIPAM165 micelles at 50° C. (ii), DMA₁₀₀-b-AAL₆₅-b-NIPAM₁₆₅ interpolyelectrolyte complexed micelles at 50° C. (iii), and DMA₁₀₀-b-AAL₆₅-b-NIPAM₁₆₅ interpolyelectrolyte complexed micelles at 25° C. (iv).

FIG. 11 depicts the Dynamic light scattering size distribution of the PEO₄₅-b-[DMA₉₈-s-NAS₃₀]-b-NIPAM₈₇ triblock copolymer under specific conditions: a) 0.5% aqueous solution at 25° C.; b) 0.5% aqueous solution at 45° C.; c) 0.5% shell cross-linked (SCL) micelle solution at 25° C.; d) At 25° C., after cleavage of the SCL micelles using Dithiothreitol (DTT); e) At 45° C., after cleavage of the SCL micelles using DTT; f) At 25° C., after re-crosslinking the cleaved SCL micelles using cystamine.

FIG. 12 depicts cumulative dipydridamole release to phosphate buffered saline solution from shell crosslinked and non-crosslinked micelles at 25° C.

FIG. 13 depicts cumulative dipydridamole release to phosphate buffered saline solution from shell cross-linked micelles with or without dithiothreitol (DTT) at 37° C.

### DETAILED DESCRIPTION OF THE PREFERRED EMBODIMENTS

One embodiment of the present invention is the controlled polymerization of AAL directly in water utilizing RAFT. In one aspect, the chain transfer agent affords excellent control of the polymerizations molecular weight and molecular weight distribution. In a preferred embodiment, the chain transfer agent is 4-cyanopentanoic acid dithiobenzoate (CTP) or 2-ethylsulfanylthiocarbonylsulfonyl-2-methyl-propionic acid (EMP). Low polydispersites (<1.19) are achieved at conversions in excess of 80%. The amino acid based monomer was then incorporated into ABC tri- and ABCBA pentablock copolymers for the facile preparation of thermally responsive, polyelectrolyte cross-linkable micelles. In one aspect, the hydrophilic block length was kept constant while the thermally responsive NIPAM block is varied to vary the temperature-dependent micellization of the block copolymers. Critical micellization temperatures (CMT) for the block copolymers are directly related to the NIPAM block length as DMA₁₀₀-b-AAL₆₅-b-NIPAM₆, exhibited a transition at 47° C. while the CMT for DMA₅₃-b-AAL₃₈-b-NIPAM₃₄₅-b-AAL₃₈-b-DMA₅₃ was 34° C. The size and molecular weight of the micelles of the present invention increases with increasing NIPAM block lengths.

In one embodiment, a cationic polymer is used for the in-situ formation of the SCL micelles via interpolyelectrolyte complexation. Preferably, the cationic polymer poly[(ar-vinyl benzyl) ammonium chloride] (PVBTAC), is employed for the in-situ formation of SCL micelles via interpolyelectrolyte complexation. The ionically cross-linked micelles of the present invention are preferably reversible in the presence of salt. The salt concentration is most preferably about 0.4 M NaCl but is less than about 1.0 M NaCl, which leads to the “salting-out” of the NIPAM block and the reformation of aggregates. The facility by which these nanocomplexes can be formed and the reversibility of these interpolyelectrolyte complexed micelle assemblies suggest that such systems may have potential application in targeted delivery and controlled release of active agents.

The present invention is also directed to a method of forming interpolyelectrolyte complexed micelles, the method comprising preparing by sequential aqueous RAFT polymerization a block copolymer comprised of N,N,-dimethyl acrylamide (DMA), N-acryloyl alanine (AAL) and N-isopropyl acrylamide (NIPAM); dissolving the block copolymers into aqueous solution; raising the solution temperature above the lower critical solution temperature of the NIPAM block; allowing the micelle solution to equilibrate; adjusting the pH of the solution to about 9.0; adding a cationic polymer to the solution; and stirring the solution.

In one embodiment the thermally responsive micelles are based on a thermo-responsive ABC triblock copolymer. In a preferred embodiment, the thermally responsive micelles are based on poly(ethylene oxide)-block-poly[(N,N-dimethylacrylamide)-stat-(N-acryloxy succinimide)]-block-poly(N-isopropyl acrylamide) copolymers which are shell crosslinked with cystamine.

In one aspect, the SCL micelles are cleaved using different types of cleaving agents. In a preferred embodiment, the cleaving agent is dithiothreitol (DTT) or tris(2-carboxyethyl)phosphine (TCEP). In one embodiment, the cleaving process is fully reversible. In a preferred embodiment of the fully reversible cleaving process, the cleaving agent is dithiothreitol (DTT) or tris(2-carboxyethyl)phosphine (TCEP). In another aspect of the invention, the SCL micelles reassemble using a thiol-exchange compound, preferably a diamine cross-linker, more preferably the cross-linker is cystamine.

In summary, triblock copolymer micelles were prepared from a novel thermo-responsive ABC triblock copolymer that self-assembles in aqueous solution. SCL micelles were readily obtained using cystamine as a diamine cross-linker. These SCL micelles can be reversibly cleaved using either DTT or TCEP; the degraded micelles can be re-crosslinked using cystamine as a thiol-exchange compound. The rate of drug release can be easily controlled from these SCL micelles, demonstrating their potential as nano-scale drug delivery vehicles.

### EXAMPLES

### Example 1

### Thermally Responsive Vesicles and their Structural “Locking” via Polyelectrolyte Complex Formation

Here we report the first example of vesicle formation from the self-assembly of hydrophilic-hydrophilic block copolymers directly in water by variation of the solution temperature. The resultant vesicles were then shell crosslinked through polyelectrolyte complexation as shown in Scheme 1.

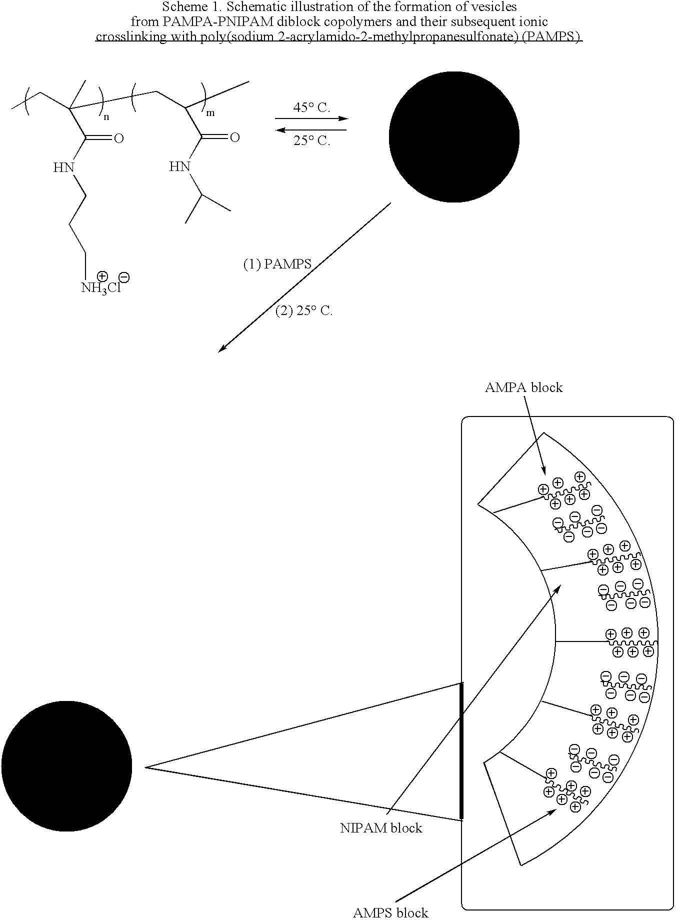

General Procedure for the RAFT Polymerization of AMPA CTP (0.0078 g, 0.028 mmol) and AMPA (1.00 g, 5.6 mmol) were added along with DI water (2.0 mL) to an ampoule. V-501 (0.00156 g, 0.0056 mmol) dissolved in 1.0 ml dioxane was then added. The solution was stirred until all the CTP was dissolved. The ampoule was sparged with nitrogen for approximately 30 min. and then placed in a preheated oil bath at 70° C. The reaction was terminated after 24 h. by cooling the reaction tube in an ice bath followed by exposure of the catalysts to air. The product was purified by dialysis against water (pH 4-5) and isolated by lyophilization.

Block Copolymer Synthesis. NIPAM (0.272 g, 2.4 mmol), PAMPA₈₈ (0.20 g) and V-501 (0.8 mg, 0.0024 mmol, dissolved in 0.6 g dioxane) were added along with DI water (0.8 mL) to an ampoule. After sparging with nitrogen for 30 min, the reaction was allowed to proceed at 70° C. for 2 h. The reaction was quenched by cooling the reaction vessel in an ice bath and exposure to air. The product was purified by dialysis against deionized water and isolated by lyophilization.

The diblock copolymer, poly(N-(3-aminopropyl)methacrylamide hydrochloride)-block-(N-isopropylacrylamide), (PAMPA-b-PNIPAM), was synthesized via the reversible addition-fragmentation chain transfer (RAFT) polymerization technique. AMPA was first polymerized in a dioxane/water mixture employing 4-cyanopentanoic acid dithiobenzoate (CTP) as the chain transfer agent (CTA) and 4,4′-Azobis(4-cyanopentanoic acid) (V-501) as the free radical source. The solution pH was maintained between 4 and 5 to avoid hydrolysis and/or aminolysis of the CTA.¹¹ Under these conditions the RAFT polymerization of AMPA is controlled. The PAMPA homopolymer was then used as macroCTA for the block polymerization of NIPAM. Adjustment of the dioxane/water ratio allows for the reaction to proceed under homogeneous condition throughout the polymerization. Well-controlled PAMPA-b-PNIPAM diblock copolymers were obtained (Table 1). The copolymer composition was calculated based on SEC and ¹H NMR data (Table 1).

TABLE 1
Block copolymers composition, molecular weight
and molecular weight distribution.

| Entry | | | |
|---|---|---|---|
| No. | Structure | Mn | Mw/Mn |
| 1 | AMPA₄₀ | 7280^{a} | 1.04 |
| 2 | AMPA₈₈ | 15700^{a} | 1.06 |
| 3 | AMPA₄₀-NIPAM₃₄ | 11300^{b} | 1.11 |
| 4 | AMPA₈₈-NIPAM₄₀ | 21200^{b} | 1.13 |
| 5 | AMPA₈₈-NIPAM₅₀ | 22500^{b} | 1.16 |
| 6 | AMPA₈₈-NIPAM₇₄ | 24100^{b} | 1.19 |

^{a}Measured by ASEC.
^{b}Measured by DMF SEC, the AMPA block was first converted to the primary amine form before SEC was conducted.

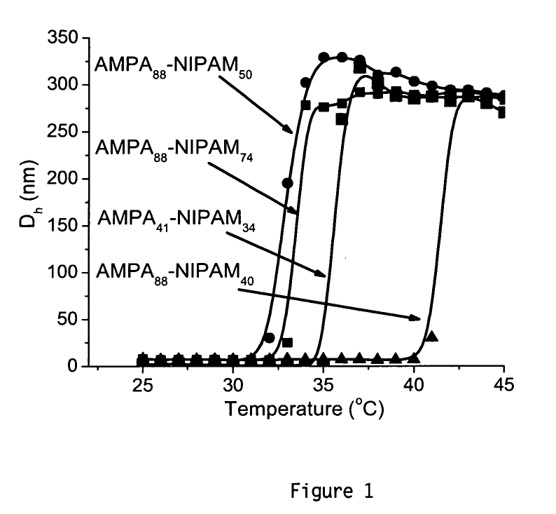

The diblock copolymers are molecularly dissolved in aqueous solution at room temperature. Increasing the solution temperature leads to the formation of uniform aggregates with diameters≈280 nm, as depicted in FIG. 1. Since the contour length of the diblock copolymers is ≈35 nm, visiclelar rather than micellar structure are found. The phase transition temperature is shown to be dependent on the block copolymer composition with polymers possessing longer NIPAM block lengths having lower phase transition temperatures. If the NIPAM block length is kept constant, increasing the AMPA block length results in an increase in the phase transition temperature. This is in agreement with recent results by Stover et al.¹² in which the lower critical solution temperature of NIPAM homopolymers is shown to be dependent on both the polymer molecular weight and hydrophilicity of the end group.

The heating rate near the phase transition temperature is an important factor on vesicle formation. In order to obtain a uniform size distribution, it was observed that the heating rate should be kept around 0.1° C./min. This was found to be especially important at higher solution concentrations (5 mg/ml). For example, at 5 mg/ml, an increase in the solution temperature from 25° C. to 45° C. over 5 min. leads to a broad size distribution. If the solution concentration is kept relatively low (<0.5 mg/ml), faster heating rates can lead to uniform vesicles, however, the vesicle sizes are smaller than those formed by a slow increase in the solution temperature. The vesicles are shown to be stable as their size remains constant above the phase transition temperature. Therefore the vesicle self-assembly process seems to be kinetically controlled.

The TEM image of the vesicles formed from the AMPA₈₈-b-NIPAM₅₀ diblock copolymer number-average particle diameter of the vesicles is approximately 145 nm, which is slightly larger than the intensity-average diameter reported by DLS (118 nm), suggesting a flattening of the vesicles adsorbed onto the TEM grid. The vesicle wall thickness is estimated to be 7 nm and is somewhat lower than the contour length of 12.5 nm that was calculated for the hydrophobic PNIPAM₅₀ block.

The vesicles are stable between pH 0 and 11. However, the particle size is shown to vary with the pH of the solution. At lower pH, the vesicle size is shown to be the largest (310 nm at pH 3.0) while increasing the solution pH decreases the vesicles size (220 nm, pH 10.8). This is most likely caused by the deprotonation of the AMPA block above its pKα. The solution concentration is also shown to influence the size distribution of the vesicles. The vesicles are larger at higher solution concentrations (283 nm, 0.5% mg/mL, 50° C.) than at lower solution concentrations (192 nm, 0.005% mg/mL, 50° C.).

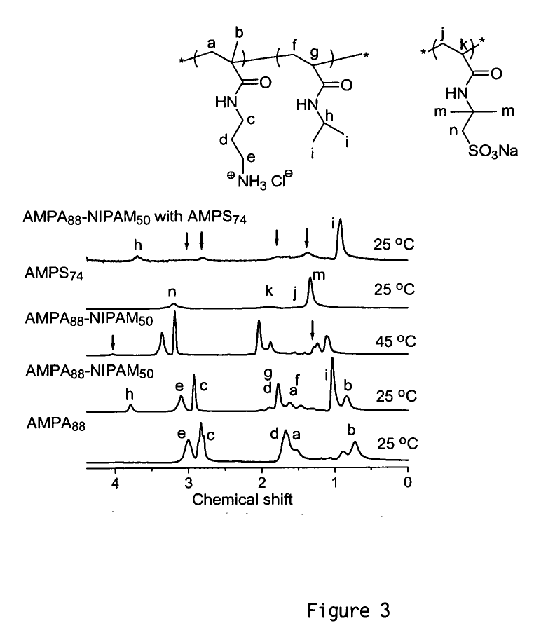

The complexation of oppositely charged polyelectrolytes has been widely used for layer-by-layer deposition and DNA condensation. Recently, Armes et al. have successfully applied this technology to form shell cross-linked micelles. ¹³ The group reported the use of a block polyelectrolyte (anionically charged) to crosslink a cationically charged micelle and concluded that a homopolyelectrolyte cross-linker only led to the formation of flocculated micelles. Here, we found that the anionic homopolymer, poly(sodium 2-acrylamido-2-methylpropanesulfonate) (PAMPS, Mₙ=17000, M_{w}/Mₙ=1.18), synthesized via RAFT, can be used to efficiently cross-link cationicaly charged vesicles. The rapid mixing of the vesicle solution with the PAMPS solution at 45° C. leads to the formation of ionically cross-linked vesicles due to the inter polyelectrolyte complexation of the PAMPA and PAMPS block (The AMPA/AMPS molar ratio was kept at 1:1). The vesicle size changes from 270 nm to 140 nm (FIG. 3) which may be attributed to a decrease in the hydrophilicity of the APMA/AMPS block upon complexation.

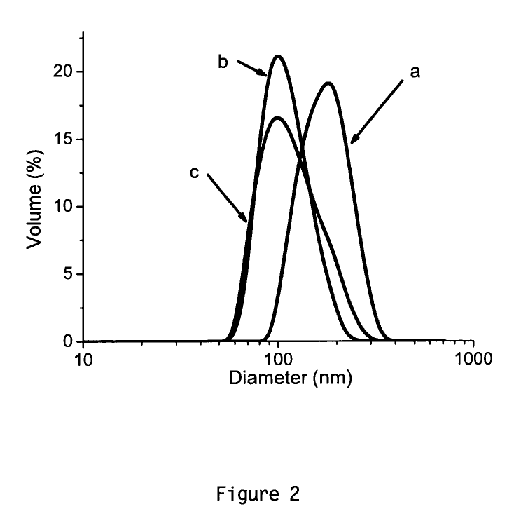

After crosslinking, the solution temperature was lowered to 25° C. DLS indicated the vesicles did not dissociate (FIG. 2), which confirmed that the vesicle structure had been successfully “locked” via polyelectrolyte complexation. This was also confirmed by ¹H NMR analysis (FIG. 3). At 25° C., the diblock copolymers are fully solvated and signals associated with each block are visible. An increase in the solution temperature to 45° C. causes the NIPAM signal to become broadened indicating the formation of vesicles (this is also confirmed by DLS shown in FIG. 1). After cross-linking at 45° C., the solution temperature was lowered to 25° C. where the NIPAM signal regains its intensity as the NIPAM block becomes hydrophilic while the AMPA and AMPS peaks broaden due to their reduced mobility caused by poleyelectrolyte complexation. It should be noted that the cross-linking process should be carried out at low concentrations (<0.5 mg/m) to prevent flocculation.

The ionically cross-linked vesicles are stable over a wide pH range (0-10.5). (Above pH 11, DLS studies indicate these particles dissociate, possibly due to hydrolytically instability.) Furthermore, the cross-linked vesicles retain their structural integrity in the presence of 0.8 M NaCl. Raising the salt concentration above 1.0 M salt causes the vesicles to dissociate.

Compared to chemical crosslinked vesicles, ionically crosslinked systems are advantageous due to the facile nature of the crosslinking reaction (the process can be completed within a few minutes) and the reversibility of the cross-links with added electrolyte, which will facilitate the removal of the vesicles after bioapplication.

### Example 2

### Responsive Nano-Assemblies via Interpolyelectrolyte Complexation of Amphiphilic Diblock Copolymer Micelles

In this example we report a facile method for the synthesis of reversible shell “locked” nano-assemblies with solution behavior idealized in Scheme 2.

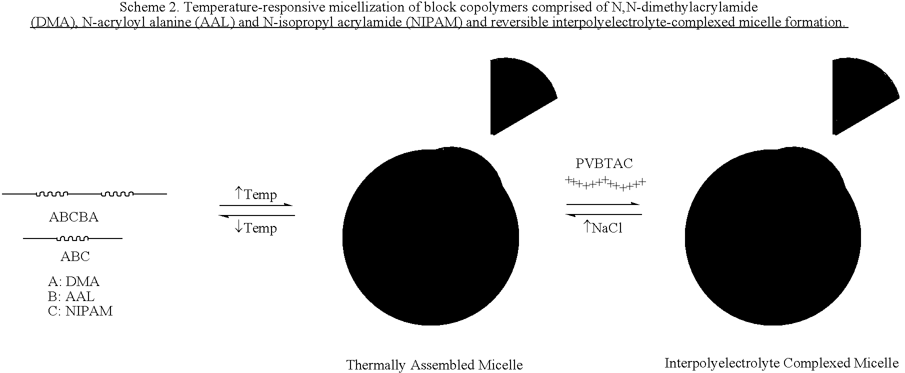

Block copolymers of N,N,-dimethyl acrylamide (DMA), N-acryloyl alanine (AAL) and N-isopropyl acrylamide (NIPAM) prepared via sequential aqueous RAFT polymerization are show to undergo a reversible, temperature-induced unimer-to-micelle transition. Alternatively, block copolymers of N,N,-dimethyl acrylamide (DMA), N-acryloyl valine (AVAL) and N-isopropyl acrylamide (NIPAM) can be used to advantage. Above the phase transition temperature, the resulting micelles are cross-linked via interpolyelectrolyte complexation of the poly(AAL) segments in the hydrated shell with the cationic homopolymer poly[(ar-vinyl benzyl) ammonium chloride] (PVBTAC). These interpolyelectrolyte complexed micelles remain intact upon cooling below the lower critical solution temperature. However, of potential technological value, is the reversibility of the crosslinking and associated loss of nanostructure integrity with addition of simple electrolytes, in our case 0.4 M NaCl. To our knowledge, this represents not only a unique method of reversible cross-linking but also the first report of the RAFT polymerization of an unprotected amino acid based monomer directly in water.

Synthesis of N-Acryloyl alanine (AAL) The synthesis of the alanine based acrylamido monomer, N-Acryloyol alanine (AAL), was performed as follows: L-Alanine (0.4 moles, 35.43 g) and NaOH (0.8 moles, 32.01 g) were dissolved in DI water (180 mL) and stirred using a mechanical stirrer. Once the solids had completely dissolved, the solution was cooled to 4° C. using an ice bath. Acryloyl chloride (0.4 moles, 36.18 g) was added drop-wise over 2 hours maintaining the temperature of the reaction at 4° C. Upon complete addition of acryloyl chloride, HCl was added dropwise to neutralize the monomer and induce precipitation. The afforded crystals were then collected by filtration and recrystallized from water. Melting point 162-164° C. (reported literature mp 163° C.)⁴⁶. ¹H NMR: CH₂CHCO 5.71 (d), CH₂CHCO 6.33 (m), HNCH(COOH)CH₃ 4.45 (m), HNCH(COOH)CH₃ 2,17 (d)

General Procedure for the RAFT Polymerization of AAL. Polymerizations were conducted at 70° C., employing V-501 as the primary radical source and CTP or EMP as the RAFT CTA. Polymerizations were performed directly in water (pH 6.5) with an initial monomer concentration ([M]₀) of 1.0 M in individual, septa-sealed vials, which were purged with nitrogen at 5° C. for 30 min prior to the reaction. The initial monomer to CTA ratio ([M]₀/[CTA]₀) was varied between 70 and 630 while the initial CTA to initiator ratio ([CTA]₀/[I]₀) was held constant at 5:1. For example, entry 3 (Table 2) was prepared by reacting 1.43 g (0.001 mol) of AAL with 39.99 mg (1.43 E-4 mols) of CTP and 8.02 mg (2.86 E-5 mols) of V-501 in 10 mL of water for 195 min. The polymerization kinetics and the absolute molecular weights were determined from aliquots (0.5 mL) taken at pre-determined time intervals and quenched via rapid cooling and exposure to oxygen.

Block Copolymer Synthesis. A macroCTA of DMA was used for preparing di- and triblock copolymers of DMA-b-AAL and DMA-b-AAL-b-DMA. The polymerizations were conducted directly in water (pH 6.5) with an initial monomer concentration of 1 M at 70° C. with V-501 as the initiator. The [PDMA]₀-[V-501]₀ ratio was maintained at 5:1, the target DP for AAL was 105. For example DMA₁₀₀-b-AAL₆₅ was prepared by reacting 1.43 g (0.001 mol) of AAL with 1.43 g (1.43 E-4 mols) of DMA₁₀₀ macroCTA and 9.26 mg (2.86 E-5 mols) of V-501 in 10 mL of water for 45 min. The DMA-b-AAL diblock and DMA-b-AAL-b-DMA triblock macroCTAs were subsequently chain extended with NIPAM to produce tri- and pentablock copolymers of DMA-b-AAL-b-NIPAM and DMA-b-AAL-b-NIPAM-b-AAL-b-NIPAM. The polymerizations were conducted directly in water (pH 5.5) with an initial monomer concentration of 1 M at 25° C. with VA-044 as the initiator. The [macroCTA]₀[VA-044]₀ ratio was maintained at 3:1, and the target degree of polymerization (DP) for NIPAM was varied. For example DMA₁₀₀-b-AAL₆₅-b-NIPAM₆₁ was prepared by reacting 1.13 g (0.001 mol) of NIPAM with 2.80 g (1.41 E-4 mols) of DMA₁₀₀-b-AAL₆₅ macroCTA and 15.24 mg (4.72 E-5 mols) of VA-044 in 10 mL of water for 180 min. Polymerizations were conducted under a nitrogen atmosphere in round-bottomed flasks equipped with magnetic stir bars and sealed with rubber septa. The products were purified by dialysis against deionized water and isolated by lyophilization.

Preparation of Micelles. Copolymers were Dissolved Directly into an Aqueous Solution (pH=6.8±0.2) containing 0.01 M NaCl (HPLC grade water) (1 mg/mL, 10 mL). Micellization of the copolymers was achieved by raising the solution temperature above the lower critical solution temperature (LCST) of the NIPAM block.

Interpolyelectrolyte Cross-Linked Micelles. Shell cross-linked micelles were prepared from an aqueous stock solution of the copolymer (1 mg/mL, 3 mL). The pH of the solution was adjusted to 9.0±0.2 and allowed to equilibrate at 50° C. for 30 min. Poly([ar-vinylbenzyl]trimethyl ammonium chloride) (PVBTAC), a cationic polymer (Mₙ=26 000 g/mol, Mₙ/M_{w}=1.21), was then added at a 1:1 mol ratio based on the carboxylic acid groups of AAL. The micelle solution was then stirred at 50° C. for 15 minutes.

Aqueous RAFT Polymerization of AAL. N-Acryloyl alanine was selected based on its facile synthesis from readily available amino acid sources and its amphoteric nature that allows polymerization, purification, and characterization directly in aqueous solution. In order to show viability of block formation, the RAFT polymerization of AAL was first performed directly in water as a control experiment using V-501 as the free radical initiator and 4-cyanopentanoic acid dithiobenzoate (CTP) or 2-ethylsulfanylthiocarbonylsulfonyl-2-methyl-propionic acid (EMP) as the chain transfer agent (CTA) (Scheme 3).

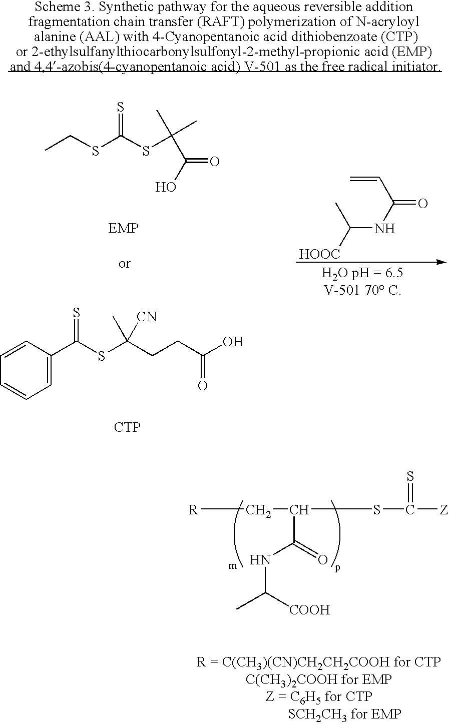

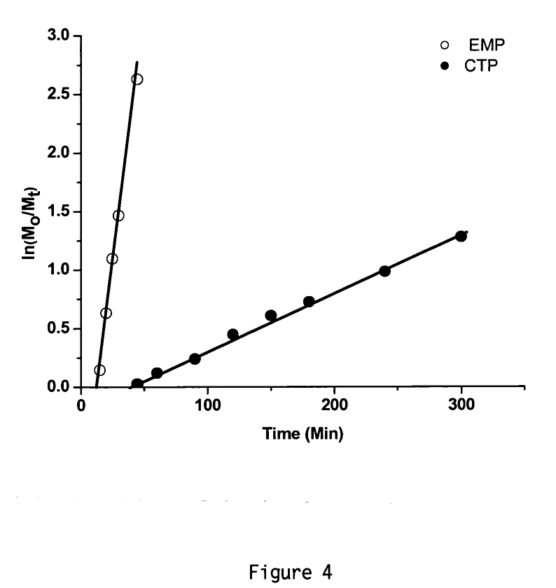

Shown in FIG. 4 are the pseudo first order kinetic plots for the CMP/EMP mediated hompolymerizations of AAL at 70° C. with an initial monomer to CTA ratio ([M]₀/[CTA]₀) of 210/1. Linear pseudo first order kinetics plots are obtained for both CTAs and the apparent rate of polymerization of EMP is significantly higher than that of CTP, the result of a higher rate of fragmentation of the intermediate radical species during the RAFT process.

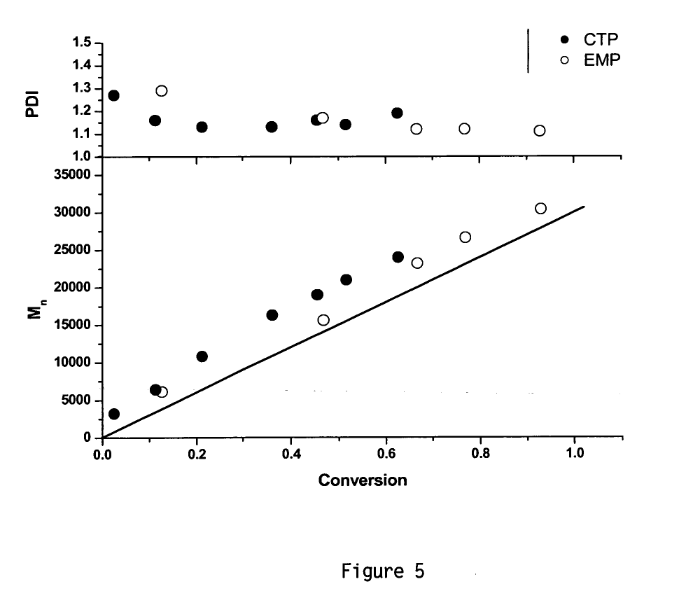

The faster rate of polymerization and the maintenance of pseudo first order kinetics to higher conversions and the closer agreement of Mₙ to theoretically predicted values, while maintaining low PDI values shown in FIG. 5 favor the use of EMP under our experimental conditions. The PDI vs conversion for the CTP/EMP polymerizations of AAL indicate that both CTAs provide good overall control of the reaction with M_{w}/Mₙ values less than 1.2 at high conversions. Given, the low polydispersities and the linear increase in molecular weight with conversion, it is clear that both CTAs allow for the controlled synthesis of AAL directly in water.

To further show the molecular weight control with our system, the initial monomer to CTA ratio ([M]₀/[CTA]₀) was varied to target degrees of polymerization of 70, 210, 420, and 630. The experimental data are summarized in Table 2 indicating that the polymers possess low PDIs (<1.3) and experimental molecular weights in good agreement with those theoretical molecular weights targeted.

TABLE 2
[Monomer]/[Chain Transfer Agent] initial concentrations ([M]₀/[CTA]₀),
percent conversion, number average molecular weight, and polydispersity
(M_{w}/Mₙ) data for the aqueous reversible addition fragmentation chain
transfer (RAFT) polymerization of N-acryloylalanine (AAL) at 70° C.
mediated by 4-Cyanopentanoic acid dithiobenzoate (CTP) and 2-ethyl-
sulfanylthiocarbonylsulfonyl2-methyl-propionic acid (EMP) with initial
[Chain Transfer Agent]:[Initiator] ratio ([CTA]₀:[I]₀) of 5:1.

| | | [M]₀/ | Time | Conv | Mn | Mnₜₕ | |
|---|---|---|---|---|---|---|---|
| Entry | CTA | [CTA]₀ | (min) | %^{b} | (g · mol⁻¹)^{c} | (g · mol⁻¹) | M_{w}/Mₙ^{c} |
| 1 | — | — | 15 | gel | — | — | — |
| 2 | CTP | 70 | 105 | 16 | 4 100 | 1 600 | 1.29 |
| 3 | CTP | 70 | 195 | 88 | 11 800 | 8 800 | 1.16 |
| 4 | CTP | 210 | 45 | 3.0 | 3 200 | 900 | 1.27 |
| 5 | CTP | 210 | 300 | 63 | 24 000 | 18 988 | 1.15 |
| 6 | CTP | 420 | 115 | 13 | 12 800 | 7 800 | 1.24 |
| 7 | CTP | 420 | 210 | 81 | 52 200 | 48 600 | 1.18 |
| 8 | CTP | 630 | 125 | 9.0 | 15 000 | 8 100 | 1.22 |
| 9 | CTP | 630 | 225 | 83 | 76 600 | 74 700 | 1.19 |
| 10 | EMP | 70 | 9 | 14 | 3 600 | 1 400 | 1.25 |
| 11 | EMP | 70 | 45 | 95 | 10 600 | 9 500 | 1.14 |
| 12 | EMP | 210 | 15 | 13 | 6 100 | 3 900 | 1.24 |
| 13 | EMP | 210 | 45 | 93 | 30 400 | 27 900 | 1.11 |
| 14 | EMP | 420 | 15 | 5.0 | 7 000 | 3 000 | 1.26 |
| 15 | EMP | 420 | 60 | 80 | 51 250 | 48 000 | 1.07 |
| 16 | EMP | 630 | 25 | 15 | 19 800 | 13 500 | 1.20 |
| 17 | EMP | 630 | 45 | 73 | 71 500 | 65 700 | 1.03 |

^{a}Polymers synthesized at 70° C. at 1 M monomer in H₂O (pH = 6.5) under a nitrogen atmosphere with V-501 as the initiator.
^{b}Conversions were determined using RI peak intensity by comparing the area of the monomer peak at time t to the area of the monomer peak at time 0.
^{c}As determined by aqueous size exclusion chromatography (ASEC)

Thermally Responsive Block Copolymers. Having established conditions for controlled/“living” polymerization of AAL, we constructed thermally responsive triblock and pentablock copolymers as detailed in the experimental section and shown in Scheme 3. The initial hydrophilic, neutral block was synthesized from DMA utilizing CMP or EMP as chain transfer agents to generate the mono-(Pathway A) or difunctional (Pathway B) macroCTAs with respective Mₙ(M_{w}/Mₙ) values of 9,900 g/mol (1.07) and 10,500 g/mol (1.04). (53) Subsequent sequential block copolymerization with AAL and then with NIPAM yielded the triblock and pentablock samples utilized in our micellization and cross-linking studies.

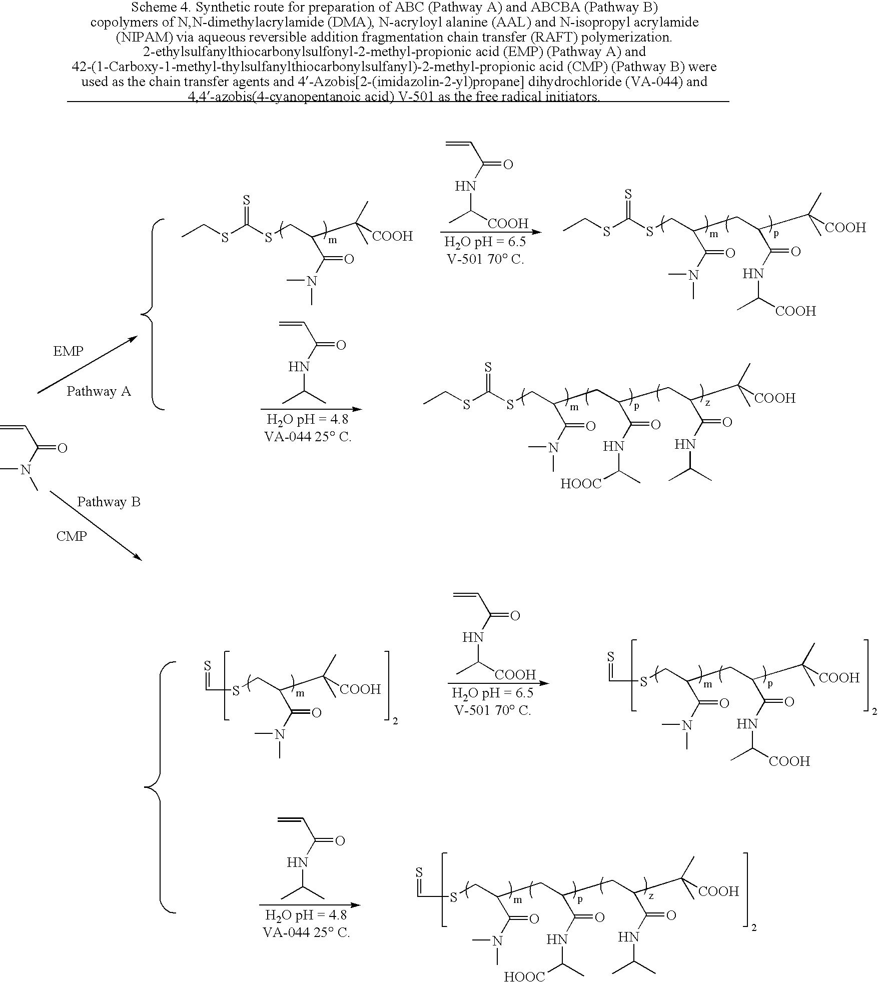

Structural data shown in Table 3 for these samples were determined by utilizing NMR and aqueous size exclusion chromatography with multi angle laser light scattering (ASEC-MALLS) detection.

TABLE 3
Degree of polymerization (DP), number average molecular weight
(Mₙ), and polydispersity (M_{w}/Mₙ) data for ABC and ABCBA copolymers
N,N-dimethylacrylamide (DMA) A block, N-acryloyl alanine (AAL)
B Block, and N-isopropyl acrylamide (NIPAM) C block.

| | DP | DP | DP | Mn | |
|---|---|---|---|---|---|
| Sample | DMA^{a} | AAL^{b} | NIPAM^{b} | (g · mol⁻¹)^{a} | M_{w}/Mₙ^{a} |
| DMA₁₀₀AAL₆₅NIPAM₆₁ | 100 | 65 | 61 | 26000 | 1.15 |
| DMA₁₀₀AAL₆₅NIPAM₁₆₅ | 100 | 65 | 165 | 37300 | 1.14 |
| DMA₅₃AAL₃₈NIPAM₁₉₄AAL₃₈DMA₅₃ | 106 | 76 | 194 | 43400 | 1.18 |
| DMA₅₃AAL₃₈NIPAM₃₄₅AAL₃₈DMA₅₃ | 106 | 76 | 345 | 60400 | 1.16 |

^{a}As determined by aqueous size exclusion chromatography (ASEC)
^{b}Determined by proton nuclear magnetic resonance (¹H NMR) spectroscopy in D₂O.

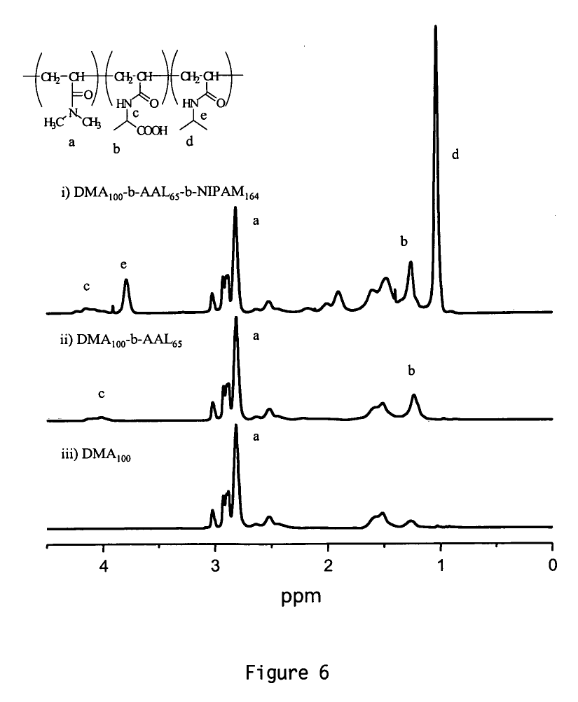

¹H NMR spectra for the DMA macroCTA and the subsequent di- and triblock copolymers are shown in FIG. 6. Block copolymer compositions were determined by comparing resonances of the DMA dimethyl group (a 2.8 ppm) to those associated with the AAL methyl (1.1 ppm) and NIPAM methine (4.2 ppm).

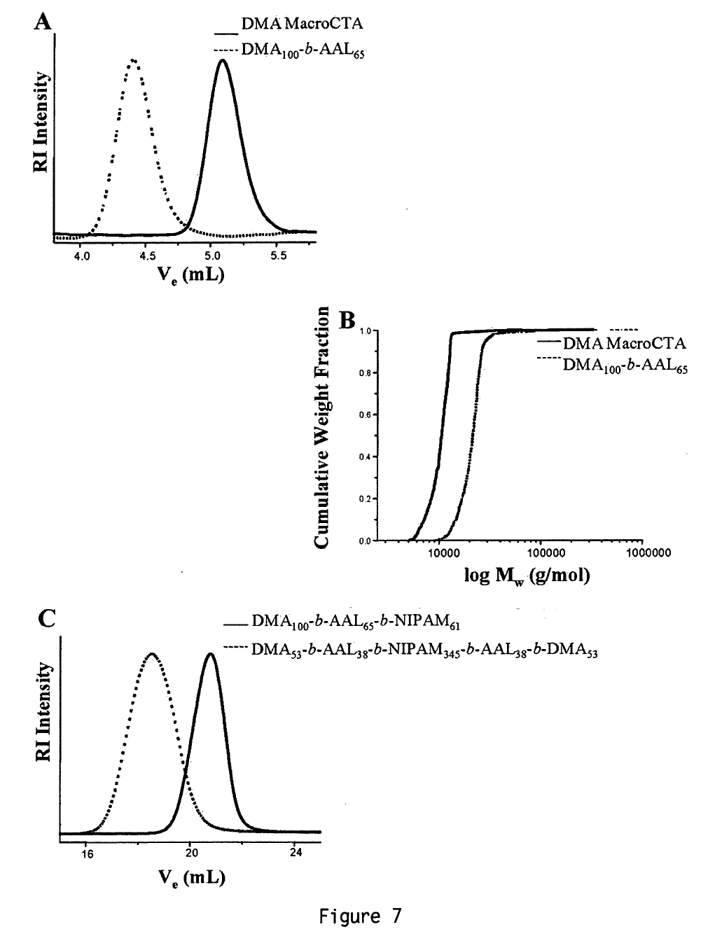

Shown in FIG. 7(A) are the normalized ASEC chromatograms and (B) cumulative weight fractions for the monofunctional DMA macroCTA and the resultant DMA₁₀₀-b-AAL₆₅ copolymer. Near-quantitative blocking efficiency (percentage of macroCTA converted to block copolymer) was confirmed by the shift in the ASEC trace (RI detector) to higher elution volume and the shift of the near-monodisperse distribution to higher molecular weight. Shown in FIG. 7 (C) are the normalized ASEC chromatograms for the ABC triblock copolymer DMA₁₀₀-b-AAL₆₅-b-NIPAM₁₆₄ and the ABCBA pentablock copolymer DMA₅₃-b-AAL₃₈-b-NIPAM₃₄₅-b-AAL₃₈-b-DMA₅₃.

ABC triblock and ABCBA pentablock copolymers with different targeted degrees of polymerization for the NIPAM containing C block (Table 3) were utilized in our experiments to demonstrate thermally reversible micellization and interpolyelectrolyte shell crosslinking (Scheme 2)

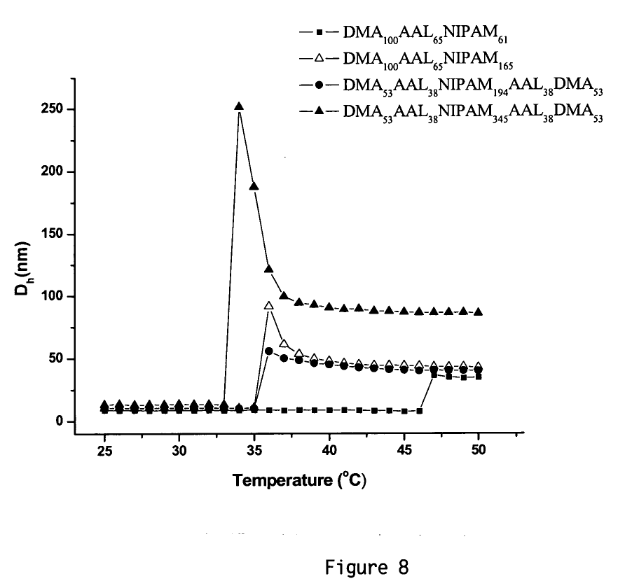

Temperature-Induced Micellization. Temperature-induced micellization can be followed using dynamic light scattering. Above its LCST, poly(NIPAM) becomes dehydrated as a result of an entropy gain resulting from the release of water molecules upon association of the isopropyl groups. (56,57) The dehydration of the NIPAM block causes the block copolymers to undergo a temperature induced transition from molecularly dissolved unimers to aggregates above the critical micelle temperature (cmt). At the cmt, a large increase in the hydrodynamic diameter of the block copolymers occurs followed by a decrease in size as the solution temperature is increased further. The decrease in micelle size with increasing temperature is most evident for the pentablock copolymer DMA₅₃-b-AAL₃₈-b-NIPAM₃₄₅-b-AAL₃₈-b-DMA₅₃ and can be attributed to its longer NIPAM block. The copolymer exists in the unimer state with an average hydrodynamic diameter of 13.2 nm below the cmt. As the temperature is further increased above the cmt, the unimers associate to form aggregates of 250 nm before reaching an equilibrium size of 85.9 nm. By contrast, DMA₁₀₀-b-AAL₆₅-b-NIPAM₆₁ shows the smallest change in hydrodynamic diameter with increasing temperature which is related to its short NIPAM block length. Here the hydrodynamic diameter of the copolymer increases from unimers of 8.8 nm to aggregates of 36.3 nm at the cmt and 34.7 nm at 50° C. Yusa and coworkers attributed this decrease in size above the cmt to either a decrease in the aggregation number of the aggregates or further dehydration of the NIPAM blocks. (58) FIG. 8 shows the temperature induced changes in the hydrodynamic volume for an aqueous solution of the respective block copolymer micelles.

The cmt for the block copolymers decreases when the NIPAM block length increases. This is evidenced by the copolymer with the shortest NIPAM block length, DMA₁₀₀-b-AAL₆₅-b-NIPAM₆₁, having the highest cmt (47° C.) while the copolymer with the largest NIPAM block length, DMA₅₃-b-AAL₃₈-b-NIPAM₃₄₅-b-AAL₃₈-b-DMA₅₃ has the lowest cmt (34° C.). These results are consistent with reports by Liu et al. (59) and Xia and coworkers (12(a)) who saw a decrease in the phase transition temperature for a series of PNIPAM homopolymers. The hydrodynamic diameter for the block copolymer series follows the trend we recently reported in which the micelle size is shown to increase with increasing NIPAM content. (53) In the current study, the micelles range from 34.7 nm for DMA₁₀₀-b-AAL₆₅-b-NIPAM₆₁ to 85.9 nm for DMA₅₃-b-AAL₃₈-b-NIPAM₃₄₅-b-AAL₃₈-b-DMA₅₃ (Table 3). The block architecture appears to have little effect on the size of the micelles as DMA₁₀₀-b-AAL₆₅-b-NIPAM₁₆₄ and DMA₅₃-b-AAL₃₈-b-NIPAM₁₉₄-b-AAL₃₈-b-DMA₅₃ has similar sizes of 42.7 nm and 39.9 nm respectively. That polydispersities of the micelles decrease with increasing NIPAM content is apparent by comparing the distribution of DMA₁₀₀AAL₆₅NIPAM₆₁ (0.153) with that of DMA₁₀₀AAL₆₅NIPAM₁₆₅ (0.075) and the distribution of DMA₅₃AAL₃₈NIPAM₁₉₄AAL₃8DMA₅₃ (0.121) with that of DMA₅₃AAL₃₈NIPAM₃₄₅AAL₃₈DMA₅₃ (0.010).

TABLE 4
Apparent hydrodynamic diameter (Dₕ), critical micelle temperature (cmt), apparent
micelle molar mass (M_{w}), and 2^{nd} virial coefficient (A₂) determined by static and
dynamic light scattering.

| | Dₕ | Dₕ | | M_{w}*10⁻⁶ | A₂ × 10⁵ |
|---|---|---|---|---|---|
| | 25° C. | 50° C. | CMT | (g*mol⁻¹) | (mL*mol/g²) |
| Sample | (nm) | (nm) | (° C.) | at 50° C. | at 50° C. |
| DMA₁₀₀AAL₆₅NIPAM₆₁ | 8.8 | 34.7 | 47 | 0.396 | 0.144 |
| DMA₁₀₀AAL₆₅NIPAM₁₆₅ | 10.4 | 42.7 | 36 | 2.18 | 8.91 |
| DMA₅₃AAL₃₈NIPAM₁₉₄AAL₃₈DMA₅₃ | 10.2 | 39.9 | 36 | 1.98 | 9.13 |
| DMA₅₃AAL₃₈NIPAM₃₄₅AAL₃₈DMA₅₃ | 13.1 | 85.9 | 34 | 7.50 | 8.55 |

Interpolyelectrolyte Cross-linking. Thermally assembled micelles were ionically cross-linked (Scheme 4) by allowing the micelle solution to equilibrate at 50° C. for 30 minutes before adding a predetermined amount (1:1 mol ratio anionic:cationic repeat units) of a 0.1% (w/w) solution of the RAFT polymerized cationic polymer PVBTAC (Mₙ=26 000 g/mol, Mₙ/M_{w}=1.21). If shell cross-linking had not occurred, dissociation into unimers would be expected upon lowering the solution to 25° C., however, DLS experiments show that micelles remain intact at room temperature thus indicating the formation of interpolyelectrolyte cross-linked micelles. The hydrodynamic diameters and zeta potentials for the SCL micelles are shown in Table 5.

TABLE 5
Apparent hydrodynamic diameter (Dₕ) and zeta potential (ξ)
for the ionically interpolyelectrolyte complexed micelles.

| | Dₕ | Dₕ | ξ (mV) 50° C. | ξ (mV) 25° C. |
|---|---|---|---|---|
| Sample | 50° C. (nm) | 25° C. (nm) | no PVBTAC | SCL |
| DMA₁₀₀AAL₆₅NIPAM₆₁ | 34.7 | 34.1 | −31.2 | −0.2 |
| DMA₁₀₀AAL₆₅NIPAM₁₆₅ | 42.7 | 37.4 | −32.9 | −2.4 |
| DMA₅₃AAL₃₈NIPAM₁₉₄AAL₃₈DMA₅₃ | 39.9 | 42.1 | −33.0 | −1.4 |
| DMA₅₃AAL₃₈NIPAM₃₄₅AAL₃₈DMA₅₃ | 85.9 | 78.6 | −38.5 | −3.4 |

In general, a slight decrease in the micelle size is observed upon allowing the solution to cool to room temperature. This is attributed to the neutralization of the AAL block which would minimize the electrostatic repulsion of the carboxyl groups and allow the chains to pack closer together. This would offset the increase in size that would be expected by the rehydration of the NIPAM blocks and result in the overall decrease of the hydrodynamic diameter of the micelles. Generally, particles with zeta potentials ≧±30 are considered stable. (60) At 50° C., the zeta potentials measured at the micelles surface (corona comprised of DMA and alanine) are in excess of −30 mV indicating the stability and net negative charge of the micelles. Upon addition of stoichiometric quantities PVBTAC to the amino acid block of the micelles, the zeta potential reached near zero values suggesting that the micelles are close to their isoelectric point (IEP). The stability of the SCL micelles was investigated using DLS over a temperature (10° C.-70° C.) and pH (2-14) range. The data indicates that the micelles are stable over the examined ranges as no dissociation into unimers is observed. The stability is attributed to the DMA corona providing sufficient steric stabilization and hydrophilicity to keep the micelles dispersed in aqueous solution. In addition, the stability/reversibility of the ionically cross-linked micelles in the presence of added salt was investigated. FIG. 6 shows the hydrodynamic diameters of the ionically cross-linked micelles and copolymer comprised of DMA₁₀₀-b-AAL₆₅-b-NIPAM1₁₆₅ in the presence of NaCl.

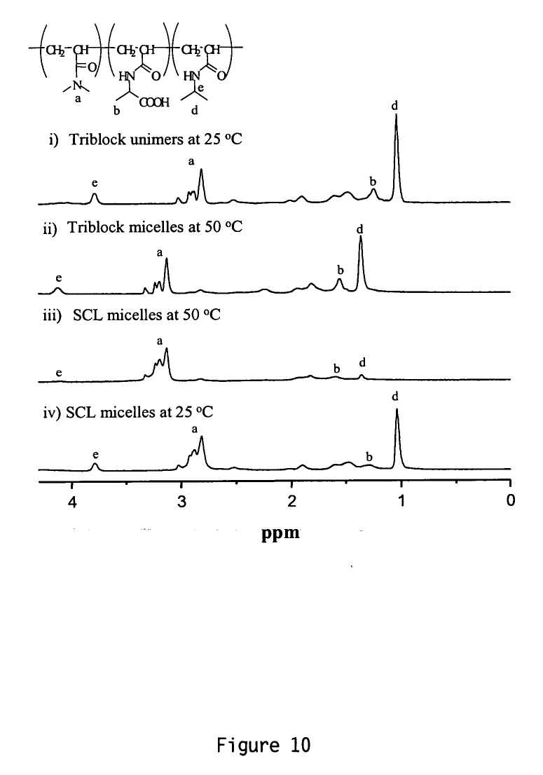

The micelles remain intact at salt concentrations as high as 0.3 M NaCl while dissociation into unimers is observed at 0.4 M NaCl demonstrating the reversibility of the ionic cross-links. It is interesting to note that at 1.0 M NaCl aggregates are once again observed. We have attributed this to the “salting-out” of the NIPAM block. To test this hypothesis, the salt response of the copolymer DMA₁₀₀-b-AAL₆₅-b-NIPAM1₁₆₅ was investigated. At 25° C. and a concentration of 0.1% w/w the diblock copolymer exists as unimers with diameters of 9.6 nm in water. Increasing the ionic strength of the solution leads to the formation of aggregates with average diameters of approximately 65 nm at 0.8 M NaCl, confirming that the addition of salt can render NIPAM blocks hydrophobic enough to induce micellization. The ¹H NMR spectra shown in FIG. 10 also support the temperature induced association and ionic cross-linking.

The transition from unimers to micelles can be followed by monitoring the changes in peak intensity with temperature. In FIG. 10, Spectrum (i) represents the triblock copolymer DMA₁₀₀-b-AAL₆₅-b-NIPAM₁₆₄ at 25° C. which is present as molecularly dissolved unimers and the peaks associated with each block are present. Upon raising the solution temperature to 50° C. (ii), the signals labeled “d” and “e” associated with the methyl and methine protons of PNIPAM become broadened and attenuated relative to the PDMA methyl signal “a” and the AAL methyl signal “b” indicating reduced mobility and solvation. This is in agreement with the DLS results which show the presence of aggregates of 42.70 nm. Upon the addition of the cationic cross-linker (iii), peak “b,” associated with the methyl group of AAL, also is broadened and attenuated relative to the PDMA methyl peak indicating its reduced mobility and solvation as a result of polyelectrolyte complexation. When the temperature of the SCL micelle solution (iii) is lowered to 25° C. (iv), an increase in intensity for the methyl and methine signals of PNIPAM, peaks “d” and “e,” while the methyl peak, “b,” of AAL remains suppressed indicating that SCL micelles have been formed.

The self-assembled morphology of the block copolymers was investigated by transmission electron microscopy (TEM). The TEM image of interpolyelectrolyte cross-linked micelles formed from the triblock copolymer DMA₁₀₀-b-AAL₆₅-b-NIPAM₆₁ clearly shows uniform micelles with diameters between 30 and 40 nm which is in reasonable agreement with that of 34.1 nm determined by DLS.

Synthesis of N-Acryloyl valine (AVAL) The synthesis of the valine based acrylamido monomer, N-Acryloyl valine (AVAL), was performed as follows: L-Valine (0.40 moles, 35.4 g) and NaOH (0.80 moles, 32.0 g) were dissolved in DI water (180 mL) and stirred using a mechanical stirrer. Once the solids had completely dissolved, the solution was cooled to 4° C. using an ice bath. Acryloyl chloride (0.40 moles, 36.2 g) was added drop-wise over 2 hours maintaining the temperature of the reaction at 4° C. Upon complete addition of acryloyl chloride, HCl was added dropwise to neutralize the monomer and induce precipitation. The afforded crystals were then collected by filtration and recrystallized from water. (Melting point 119-120° C.; ¹H NMR: CH₂CHCO 5.71 (d), CH₂CHCO 6.22 (m), HNCH(COOH)CH(CH₃)₂ 4.15 (d), HNCH(COOH)CH(CH₃)₂ 2.01 (m), HNCH(COOH)CH(CH₃)₂ 0.98 (d))

General Procedure for the RAFT Polymerization of AVAL. Polymerizations were conducted at 30° C. or 70° C., employing 4,4′-Azobis dihydrochloride (VA-044) or 4,4′-Azobis(4-cyanopentanoic acid) (V-501) as the primary radical source and 2-Ethylsulfanylthiocarbonylsulfonyl-2-methyl-propionic acid EMP as the RAFT CTA. Polymerizations were performed directly in water (pH 6.5) with an initial monomer concentration ([M]₀) of 1.00 M in individual, septa-sealed vials, which were purged with nitrogen at 5° C. for 30 min prior to reaction. The initial monomer to CTA ratio ([M]₀/[CTA]₀) was 210 while the initial CTA to initiator ratio ([CTA]₀/[I]₀) was held constant at 5:1. For example, to determine the kinetics for the RAFT polymerization of AVAL mediated by EMP at 70° C., 5.00 g (0.029 mol) of AVAL, 31.4 mg (1.40 E⁻⁴ mols) of EMP, and 7.85 mg (2.80 E-5 mols) of V-501 were added to 29 mL of DI water. The polymerization kinetics and the absolute molecular weights were determined from aliquots (0.5 mL) taken at pre-determined time intervals and quenched via rapid cooling and exposure to oxygen.

Block Copolymer Synthesis. A macroCTA of DMA was used for preparing diblock copolymers of DMA-b-(NIPAM-s-AVAL). The polymerizations were conducted directly in water (pH 6.5) with an initial monomer concentration of 1.00 M at 30° C. with VA-044 as the initiator. The [PDMA]₀-[VA-044]₀ ratio was maintained at 5:1, the target Mₙ for the second block was 25,000 g/mol. The feed ratio for AVAL was varied between 10 and 40 mol %. For example, DMA₁₁₅-b-(NIPAM₂₀₂-s-AVAL₂₇) was prepared by adding 1.09 g (0.009 mol) of NIPAM, 0.171 g (0.001 mol) of AVAL, 0.516 g (4.53 E-3 mol) of DMA₁₁₅ MacroCTA and 2.93 mg (9.05 E-4 mols) of VA-044 to 10 mL of DI water and allowing the polymerization to proceed for 360 min. Polymerizations were conducted under a nitrogen atmosphere in round-bottomed flasks equipped with magnetic stir bars and sealed with rubber septa. The products were purified by dialysis against deionized water and isolated by lyophilization.

Preparation of Micelles. Copolymers were Dissolved Directly in HPLC Grade Water containing 0.01 M NaCl (1 mg/mL, 10 mL), and the pH was adjusted using 0.1 N HCl or NaOH. Micellization of the copolymers was achieved by adjusting both the solution pH and temperature.

Interpolyelectrolyte Cross-Linked Micelles. Shell cross-linked micelles were prepared from an aqueous stock solution of the copolymer (1 mg/mL, 3 mL). The pH of the solution was adjusted to 4.2±0.2 equilibrated at 50° C. for 30 min. PVBTAC, a cationic polymer (Mₙ=26 000 g/mol, Mₙ/M_{w}=1.21), was then added at a 1:1 mol ratio based on the deprotonated carboxylic acid groups of AVAL. The micelle solution was then stirred at 50° C. for 15 minutes.

Aqueous RAFT Polymerization of AVAL. N-Acryloyl valine, a structural isomer of the pH responsive monomer 3-acrylamido-3-methylbutanoate (AMBA) was selected for this study based on its facile synthesis from readily available amino acid sources and its amphiphilic nature, which allows for polymerization, purification, and characterization directly in aqueous solution. In order to incorporate the monomer into pH- and temperature-responsive block copolymers, the RAFT polymerization of AVAL directly in water was investigated utilizing the diazo initiators V-501 (70° C.) or VA-044 (30° C.) and 2-ethylsulfanylthiocarbonylsulfonyl-2-methyl-propionic acid (EMP) as the chain transfer agent (CTA) (Scheme 5).

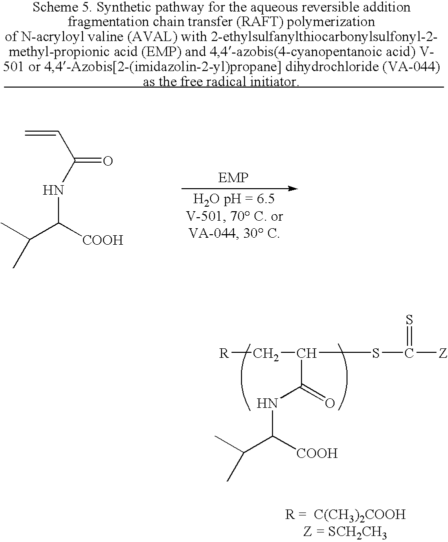

The reaction data showed pseudo first order kinetic plots for the EMP mediated hompolymerizations of AVAL at 30° C. and at 70° C. with an initial monomer to CTA ratio ([M]o[CTA]₀) of 210:1. The apparent rate of polymerization at 70° C. is significantly higher than at 30° C. This is attributed to a higher fragmentation rate for the intermediate radical species at 70° C. In addition, it should be noted that an induction period (˜5 hrs) is observed for the RAFT polymerization of AVAL at 30° C.

The linear increases in molecular weight versus conversion, excellent agreement between theoretical (solid line) and experimental molecular weights, and low polydispersities for the aqueous RAFT polymerization of AVAL at 30° C. and 70° C. indicate that the RAFT mediated polymerization of AVAL at both 30 and 70° C. proceeds in a controlled fashion. Of particular interest is the ability to control the polymerization of AVAL 30° C., which allows for copolymerization with a temperature-responsive monomer, such as NIPAM, directly in water.

pH and Temperature Responsive Block Copolymers. Having established conditions for the controlled polymerization of AVAL, pH- and temperature-responsive diblock copolymers were synthesized as detailed in the experimental section and shown in Scheme 6. The initial hydrophilic, neutral block synthesized from DMA utilizing EMP as the chain transfer agent had an Mₙ of 11,400 g/mol and a PDI of 1.07. Subsequent block copolymerization with NIPAM and AVAL yielded the pH- and temperature-responsive diblock copolymers utilized for micellization studies.

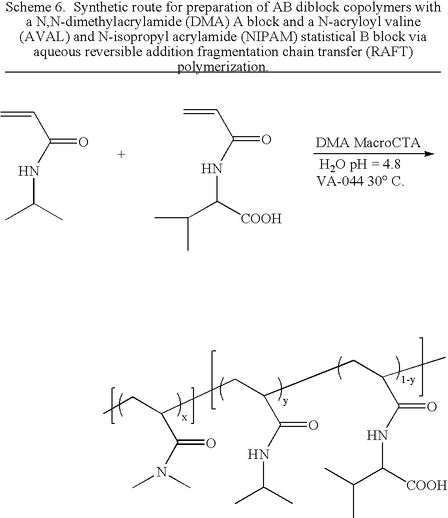

The structural data for the copolymers (Table 6) were determined utilizing aqueous size exclusion chromatography with multi angle laser light scattering (ASEC-MALLS) detection and ¹H NMR.

TABLE 6
Degree of polymerization (DP) for N,N,-dimethylacrylamide (DMA),
N-isopropyl acrylamide (NIPAM), and N-acryloyl valine (AVAL,
NIPAM/AVAL mole percents, molecular weight (Mₙ), and polydispersity
(PDI) for DMA₁₁₅-b-NIPAM-s-AVAL block copolymers 1-5.

| | DP | DP | DP | NIPAM/AVAL | | |
|---|---|---|---|---|---|---|
| Entry | DMA^{a} | NIPAM^{b} | AVAL^{b} | Mol %^{b} | M_{N}^{b} | PDI^{a} |
| 1 | 115 | 252 | 0 | 100/0 | 39,900 | 1.18 |
| 2 | 115 | 202 | 27 | 88/12 | 38,800 | 1.19 |
| 3 | 115 | 182 | 47 | 79/21 | 39,900 | 1.15 |
| 4 | 115 | 161 | 67 | 71/29 | 41,000 | 1.13 |
| 5 | 115 | 131 | 80 | 62/38 | 39,900 | 1.16 |

^{c}As determined by aqueous size exclusion chromatography (ASEC)
^{d}Determined by proton nuclear magnetic resonance (¹H NMR) spectroscopy in D₂O.

Temperature-Induced Micellization. Dynamic light scattering was utilized to study the pH- and temperature-induced assembly (micellization) of the block copolymers shown in Table 6. The unimer-to-micelle transition was followed using dynamic light scattering. The CMT for the control, a diblock copolymer of DMA₁₁₅-b-NIPAM₂₅₂ was determined to be 35-36° C. By copolymerizing 10-40 mol % AVAL into the NIPAM block and adjusting the pH of the solution to 2, the CMT of the block copolymers is shifted to significantly lower temperatures. The CMTs for the diblock copolymers at pH 2 range from 23° C. for DMA₁₁₅-b-(NIPAM₂₀₂-S-AVAL₂₇) (10 mol % AVAL) to 9° C. for DMA₁₁₅-b-(NIPAM₁₃₁-s-AVAL₈₀) (40 mol % AVAL).

Similar trends were observed at pH 4 where roughly 50% of the AVAL units are deprotonated. The CMTs range from 32° C. for DMA₁₁₅-b-(NIPAM₂O₂₋ₛ-AVAL₂₇) (10 mol % AVAL) to 28° C. for DMA₁₁₅-b-(NIPAM161-s-AVAL₆₇) (30 mol %). It should be noted that the range is much smaller at pH 4 than at pH 2 and that DMA₁₁₅-b-(NIPAM₁₆₁-s-AVAL₆₇) (30 mol %) actually has the lowest CMT of 28° C. At pH 8, essentially all of the AVAL units are deprotonated and impart enough hydrophilicty to the statistical block to suppress the hydrophobic interactions of PNIPAM above its LCST, thus preventing micellization. The CMTs for the diblock copolymers at pH values 2, 4, and 8 and the apparent Dₕ values below and above the CMT are summarized in Table 7.

TABLE 7
Critical micelle temperature (CMT) values at pH 2, 4, and 8 and
apparent hydrodynamic diameter (Dₕ) (nm) at T < CMT, and Dₕ (nm)
at T > CMT for DMA₁₁₅-b-NIPAM₂₅₂ (entry 1),
DMA₁₁₅-b-(NIPAM₂₀₂-s-AVAL₂₇) (entry 2),
DMA₁₁₅-b-(NIPAM₁₈₂-s-AVAL₄₇) (entry 3),
DMA₁₁₅-b-(NIPAM₁₆₁-s-AVAL₆₇) (entry 4), and
DMA₁₁₅-b-(NIPAM₁₃₁-s-AVAL₈₀) (entry 5)
measured by dynamic light scattering
(polymer concentration = 1.0 g/L).

| | CMT (° C.) | | | Dₕ (nm) T < CMT | | | Dₕ (nm) T > CMT | | |
|---|---|---|---|---|---|---|---|---|---|
| Entry | pH 2 | pH 4 | pH 8 | pH 2 | pH 4 | pH 8 | pH 2 | pH 4 | pH 8 |
| 1 | 35 | 36 | 36 | 10.2 | 10.3 | 10.1 | 49.4 | 49.5 | 49.1 |
| 2 | 23 | 32 | NT | 10.2 | 6.65 | 7.66 | 52.2 | 67.7 | 7.66 |
| 3 | 14 | 30 | NT | 10.0 | 6.81 | 7.34 | 49.0 | 82.3 | 7.34 |
| 4 | 11 | 28 | NT | 10.4 | 6.28 | 6.99 | 44.9 | 85.8 | 6.99 |
| 5 | 9 | 29 | NT | 10.0 | 6.62 | 7.13 | 48.9 | 73.2 | 7.13 |

(NT = no observed CMT)

Combined pH and Temperature Effects on Micelle Size. In general, increasing the solution pH leads to an increase in the copolymer CMT value up to a critical pH at which micellization does not occur. A large increase in the apparent hydrodynamic diameter of the assemblies is observed between pH 4 and 5. This is attributed to the deprotonation of the AVAL units which increases electrostatic repulsions and chain stretching. Above pH 5, a sufficient number of the AVAL units are deprotonated and impart enough hydrophilicity to prevent association.

Interpolyelectrolyte Cross-linking. The thermally assembled entities were ionically cross-linked by allowing the micelle solution (pH 4.2±0.2) to equilibrate at 50° C. for 30 min before adding a predetermined amount (1:1 mol ratio anionic:cationic repeat units) of a 0.1% (w/w) solution of the RAFT polymerized, poly(ar-vinylbenzyl) trimethyl ammonium chloride (PVBTAC) (Mₙ=26 000 g/mol, Mₙ/M_{w}=1.21). DLS indicates that the micelles remain intact upon lowering the solution to 25° C., indicating the formation of interpolyelectrolyte cross-linked micelles.

At 25° C. DMA₁₁₅-b-(NIPAM₁₆₁-s-AVAL₆₇) polymers exists as unimers with an apparent Dₕ of 7.0 nm. Raising the temperature to 50° C. induces the formation of micelles with average apparent Dₕ values of 96.9 nm. Interpolyecletrolyte complexed micelles are formed through the addition of an equimolar (anionic:cationic) amount of PVBTAC to the micelle solution at 50° C. This is accompanied by a decrease in the apparent Dₕ to 52.4 nm due to charge neutralization. “Locking” of the self-assembled structures is evidenced by the existence of micelles below the LCST of the NIPAM segments which are “swollen” to apparent Dₕ values of 57.2 nm after lowering the solution temperature to 25° C. The addition of 0.3 M NaCl is then shown to induce dissociation of the complexed micelles into their unimeric components with an average apparent Dₕ of 7.7 nm.

In this example we have shown the controlled polymerization of AVAL directly in water utilizing RAFT. Excellent control over the molecular weight and molecular weight distribution can be achieved at both 30° C. and 70° C. The amino acid based monomer AVAL was then copolymerized with NIPAM to form pH and temperature responsive block copolymers. The hydrophilic DMA block length was kept constant while 10-40 mol % of AVAL was copolymerized with NIPAM in order to synthesize a series of pH-triggered, temperature-responsive block copolymers and to investigate their self-assembly behavior. It was shown that the critical micellization temperature for the block copolymers can be tuned by adjusting the solution pH. Micelles with apparent hydrodynamic diameters ranging from 45 to 86 nm with LCST values between 9 and 36° C. were formed at pH values between 2 and 5. Above pH 5.5, a sufficient number of the AVAL units are deprotonated and impart enough hydrophilicity to the block compolymers to prevent micellization. Additionally, it was shown that reversible polyelectrolyte complexed micelles could be formed through the addition of the cationic polymer PVBTAC. These interpolyelectrolyte “locked” micelles are stable in aqueous solution at room temperature (below LCST) but complexation can be reversed by addition of sufficient electrolyte. The facility by which these nanocomplexes can be formed, the ability to tune the CMT by adjusting the solution pH, and the reversibility of these interpolyelectrolyte complexed micelle assemblies suggest that such systems may have potential applications in targeted delivery and controlled release of active agents, e.g. hydrophobic drug moieties.

### Example 3

### Synthesis of Reversible Shell Cross-Linked Micelles for Controlled Release of Bioactive Agents

The key features of micelle assembly, reversible cross-linking and thermo-responsive behavior are illustrated in Scheme 7.

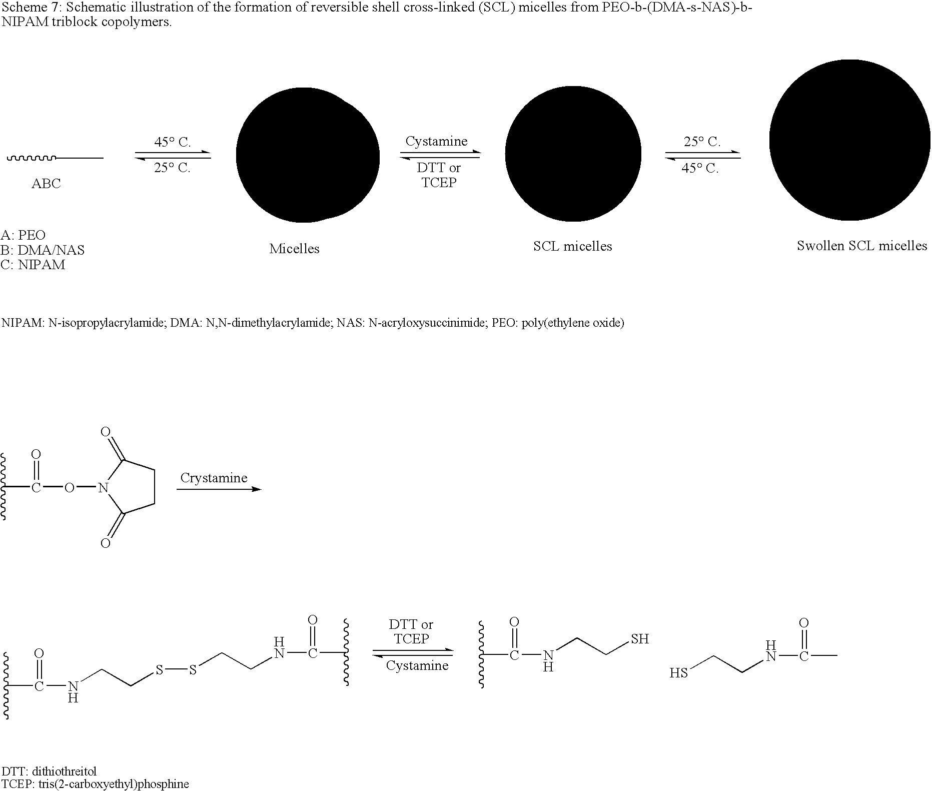

PEO-b-(DMA-s-NAS) Copolymers. Having established a suitable polymerization procedure for the homopolymerization of DMA from the PEO macro-CTA, we then examined the incorporation of an active monomer by the statistical copolymerization of DMA with NAS. NAS was chosen as the active monomer due to its enhanced activity toward primary amines and relatively lower susceptibility to hydrolysis.⁵² The copolymerization of NAS with other vinyl monomers via RAFT has already been reported^{52,53} although the homopolymerization of NAS by RAFT is uncontrolled.⁵⁴ Such polymer precursors have been used as supports for oligonucleotide synthesis, to elaborate polymer-oligonucleotide conjugates, or used as active sites in the synthesis of comblike polymers.⁵⁶ Our results for the RAFT copolymerization of DMA with NAS in 1,4-dioxane yielded the following structures:

PEO₄₅-b-(DMA₉₁₋ₛ-NAS 12)

PEO₄₅-b-(DMA₉₂₋ₛ-NAS₃₁)

PEO₄₅-b-(DMA₄₀-s-NAS₅₃)

PEO₄₅-b-(DMA₉₁₋ₛ-NAS₁₂)-b-NIPAM₅₇

PEO₄₅-b-(DMA₉₁₋ₛ-NAS₁₂)-b-NIPAM₈₈

PEO₄₅-b-(DMA₉₂₋ₛ-NAS₃₁)-b-NIPAM₅₃

PEO₄₅-b-(DMA₉₂₋ₛ-NAS₃₁)-b-NIPAM₉₁

The polydispersities of the copolymers are relatively narrow (<1.2), indicating good control of the polymerization. To obtain the copolymer compositions, the NAS moieties were reacted with excess amounts of benzylamine.

PEO-b-(DMA-s-NAS)-b-NIPAM Triblock Copolymers NIPAM is an extensively studied nonionic, acrylamido monomer since its homopolymer (PNIPAM) possesses a readily accessible LCST of 32° C. in aqueous solution, close to human body temperature (37° C.). For this reason, PNIPAM has been evaluated in drug delivery applications.⁵⁷⁻⁵⁹ Several groups have already reported the successful RAFT polymerization of NIPAM.⁶⁰⁻⁶³ We used PNIPAM as the third block in our ABC triblock copolymers. The ABC triblock copolymer was synthesized using the PE045-b-(DMA-s-NAS) as the macro-CTA in the RAFT polymerization of NIPAM. All synthetic triblock copolymers have relatively low polydispersities (<1.3), and the chain transfer efficiency is high since there appear to be no low molecular weight side peaks which correspond to the PEO-b-(DMA-s-NAS) diblock macro-CTA

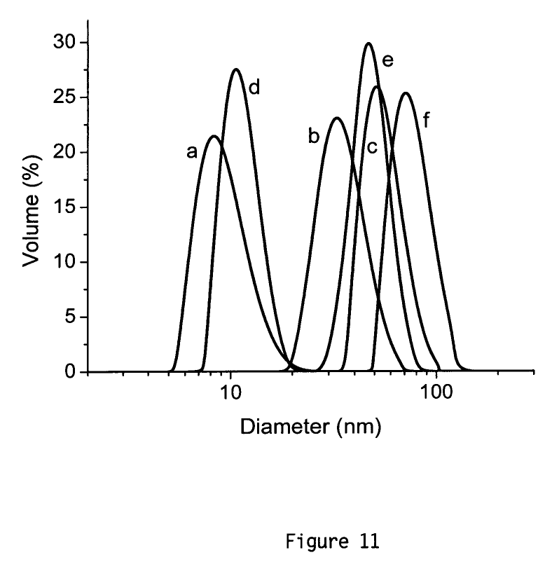

The triblock copolymer, poly(ethylene oxide)-block-[(N,N-dimethylacrylamide)stat-(N-acryloxysuccinimide)]-block-(N-isopropylacrylamide), [PEO-b-P(DMA-stat-NAS)-b-NIPAM], was synthesized in dioxane at 70° C. using a poly(ethylene oxide)-based chain transfer agent (macroCTA) using a reversible addition-fragmentation chain transfer polymerization protocol. (65) Size exclusion chromatography and ¹H NMR analysis confirm the triblock copolymer structure to be PEO₄₅-b-[DMA₉₈₋ₛ-NAS₃₀]-b-NIPAM₈₇. At room temperature this triblock copolymer is molecularly dissolved and has a hydrodynamic diameter of approximately 7-8 nm (FIG. 11, a). Increasing the solution temperature to 37° C. leads to the formation of micelles with PNIPAM cores. Spherical micelles with hydrodynamic diameters of 38 nm are formed at 45° C. The NAS unit in the center block of the triblock copolymer was then cross-linked using 1 molar equivalent of cystamine, a disulfide-based bifunctional primary amine. Dynamic light scattering (DLS) studies indicated that the micelle size did not change during cross-linking (FIG. 11, b). After cross-linking, the solution temperature was lowered to room temperature. DLS results showed an increase in the micelle diameter to 57 nm (FIG. 11, c). This larger micelle size is due to swelling of the micelle cores as the core-forming PNIPAM chains become hydrophilic under these conditions.

Disulfide bonds can be easily cleaved by Tris(2-carboxyethyl)phosphine (TCEP). In the presence of 0.05 mol/L TCEP, the SCL micelles are cleaved to produce individual copolymer chains within 30 min. at room temperature. The disulfide bonds can also be cleaved by a well-known thiol-disulfide exchange reaction using compounds such as dithiothreitol (DTT). DLS studies confirmed that all SCL micelles can be completely de-crosslinked at 45° C. using excess DTT after 10 h (FIG. 11, d). The mean hydrodynamic diameter decreased from 60 nm to 9-10 nm, which is comparable to that of the triblock copolymer precursor. Disulfide-cleavage was also confirmed by SEC analysis; the retention volume of the DTT-treated SCL micelles is significantly higher than that of the SCL micelles, but it is in the same range as that of the triblock copolymer precursor. In both cases the excess TCEP or DTT and their corresponding by-products can be removed by dialysis.

After cleavage with either TCEP or DTT, the disulfide-containing cystamine groups of the triblock copolymer are converted to thiol units and the triblock copolymer chains retain their thermo-responsive character. Micelles are re-formed when the solution temperature is raised to 45° C. (FIG. 11, e, 50 nm). These micelles are slightly larger than those formed by the original PEO-b-[DMA-stat-NAS]-b-NIPAM triblock copolymer (FIG. 11, f). A possible explanation is that, after cleavage, the middle block becomes slightly more hydrophilic than the original DMA-stat-NAS block, thus increasing the micelle size. These micelles can be re-crosslinked using a further charge of cystamine. In contrast to the original crosslinking of the NAS units by the cystamine residues, this second charge of cystamine acts as a thiol/disulfide exchange reagent. DLS studies confirm that there was no significant size change during the re-formation of the SCL micelles. After re-crosslinking, the solution temperature was lowered to room temperature. DLS indicated a mean particle diameter of about 75 nm (FIG. 11, f), which confirmed that cross-linking had been successful (otherwise micelle dissociation would have occurred below the LCST of the PNIPAM chains). This is also confirmed by TEM analysis. The final degree of crosslinking of the re-formed SCL micelles depends on the precise conditions (e.g. micelle concentration, cystamine concentration, reaction temperature and time) selected for the thiol/disulfide exchange reaction. After thiol/disulfide exchange, the chemical structure of the re-formed SCL micelles is the same as that before cleavage (Scheme 5). Thus the SCL micelles can be uncrosslinked and re-crosslinked repeatedly, in a fully reversible process.

Dipydridamole (DIP) was used as a model compound for controlled release studies using these novel SCL micelles. Solution A: The PEO-b-(DMA-s-NAS)-b-NIPAM triblock copolymer (50 mg) was dissolved in H₂O (4.0 ml) at room temperature, and this solution was placed in a 45° C. water bath. The solution was equilibrated for 10 min. to allow the formation of micelles. Solution B: A solution of cystamine (6.3 mg) dissolved in 3.0 mL H₂O (pH 9.0) was placed in a water bath at 45° C. and allowed to equilibrate for 10 min. 10.0 mg DIP was added to 4.0 ml H₂O and 0.050 ml of a 1 M HCl solution was then added to fully dissolve the DIP. This solution was placed in a 45° C. water bath. After 10 min. 0.050 ml of a 1 M NaOH solution was added to adjust the solution pH to 7, causing the DIP to precipitate. Solution A (45° C.) was then added, causing the DIP to redissolve. Solution B (45° C.) was then added to the solution. The final copolymer concentration was 5 g/L and the cystamine/NAS molar ratio was kept constant at 1:2. The solution was stirred for 3.5 hours at 45° C.

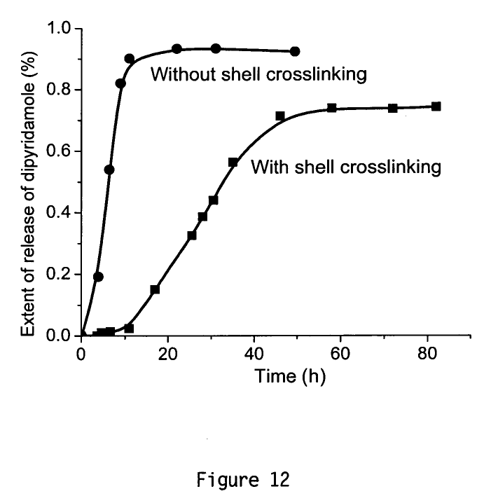

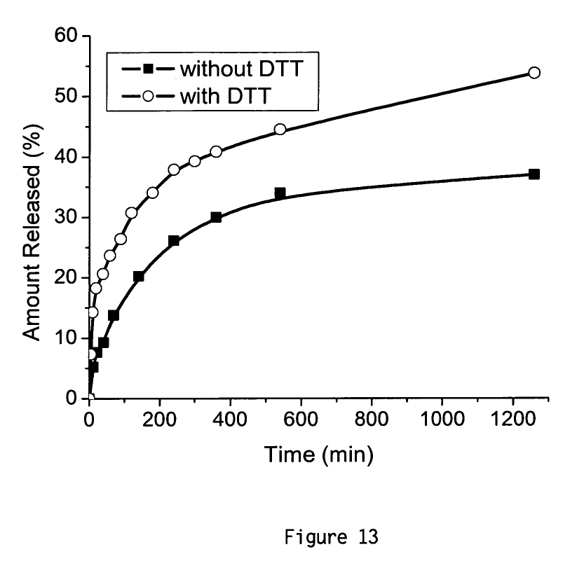

By mixing DIP with the thermo-responsive micelles at 45° C., the DIP can be loaded into the core of the micelles. Subsequently lowering of the solution temperature to 25° C. elicits micelle dissociation, which leads to triggered release of the DIP. The rate of release can be directly monitored by visible absorption spectroscopy at 415 nm, which is the characteristic absorption for DIP. Shell crosslinking the DIP-loaded micelles using cystamine can significantly retard the rate of release (FIG. 12). The release of DIP could not be detected after 7 days, meaning that the remaining DIP was stabilized within the core of the SCL micelles. Drug release from DIP-loaded SCL micelles was also monitored by visible absorption spectroscopy using a conventional dialysis method as shown in FIG. 13. The release rate is much faster in the presence of DTT (which causes cleavage of the SCL micelles, therefore the diffusion of the DIP from the core of the micelles to the environmental PBS buffer will be facile). Since the rate of cleavage of the SCL micelles can be controlled by using different types of chemical agents (such as TECP or DTT) or by adjusting their concentration or reaction temperature, the release rate of the DIP can be easily controlled. The SCL micelles can also be cleaved by using an oxidizing agent such as H₂O₂, (68d) which leads to complete cleavage of the SCL micelles within 10 h at 60° C. Since H₂O₂ is produced in the mammalian immune system, in-situ cleavage of the SCL micelles should be feasible, possibly facilitating the elimination of the individual copolymer chains after drug delivery via renal excretion.

### REFERENCES

[1] H. R. Petty, Molecular Biology of Membranes, Structure and Function; Plenum; New York, 1993.
[2] (a) L. Zhang, A. Eisenberg, Science 1995, 268, 1728; (b) D. E. Discher, A. Eisenberg, Science 2002, 297, 967; (c) L. Zhang, A. Eisenberg, Macromolecules 1996, 29, 8805; (d) L. Luo, A. Eisenberg, Langmuir 2001, 17, 6804; (e) L. Luo, A. Eisenberg, Angew. Chem., Int. Ed. 2002, 41, 1001-1004. (f) J. Wu, A. Eisenberg, J. Am. Chem. Soc. 2006, 128(9), 2880-2884.
[3] B. M. Discher, Y. Won, D. S. Ege, J. C. M. Lee, F. S. Bates, D. E. Discher, D. A. Hammer, Science 1999, 284, 1143.
[4] P. P. Ghoroghchian, G. Li, D. H. Levine, K. P. Davis, F. S. Bates, D. A. Hammer, M. J. Therien, Macromolecules 2006, 39(5), 1673 (b) Y. He, Z. Li, P. Simone, T. P. Lodge, J. Am. Chem. Soc. 2006, 128(8), 2745-2750.
[5] (a) J. Z. Du, Y. M. Chen, Y. H. Zhang, C. C. Han, K. Fischer, M. Schmidt, J. Am. Chem. Soc. 2003, 125, 14710 (b) J. Z. Du, Y. M. Chen, Macromolecules 2004, 37, 5710 (c) J. Z. Du, Y. M. Chen, Angew. Chem., Int. Ed. 2004, 43, 5084 (d) J. Z. Du, Y. M. Chen, Macromolecules 2004, 37, 6322.
[6] P. L. Soo, A. Eisenberg, J. Polym. Sci. Polym. Phys. 2004, 42, 923.
[7] (a) F. Checot, S. Lecommandoux, Y. Gnanou, H. A. Klok, Angew. Chem., Int. Ed. 2002, 43, 1339 (b) J. Rodriguez-Hernandez, S. Lecommandoux, J. Am. Chem. Soc. 2005, 127, 2026.
[8] (a) J. Du, S. P. Armes, J. Am. Chem. Soc. 2005; 127(37), 12800 (b) J. Du, Y. Tang, A. L. Lewis, S. P. Armes, J. Am. Chem. Soc,; 2005, 127(51), 17982.
[9] C. L. McCormick, A. B. Lowe, Acc. Chem. Res. 2004, 37, 312.
[10] (a) Y. Li, B. S. Lokitz, C. L. McCormick, Macromolecules, 2006; 39(1); 81-89. (b) Y. Li, B. S. Lokitz, S. P. Armes, C. L. McCormick, Macromolecules, 2006, 39(8), 2726.
[11] D. B. Thomas, A. J. Convertine, R. D. Hester, A. B. Lowe, C. L. McCormick, Macromolecules,; 2004, 37(5), 1735.
[12] (a) Y. Xia, X. Yin, N. A. D. Burke, H. D. H. Stover, Macromolecules., 2005, 38(14), 5937-5943. (b) Y. Xia, N. A. D. Burke, H. D. H. Stover, Macromolecules; 2006, 39(6), 2275.
[13] J. V. M. Weaver, Y. Q. Tang, S. Liu, P. D. Iddon, R. Grigg, S. P. Armes, N. C. Billingham, R. Hunter, S. P. Rannard, Angew. Chem., Int. Ed. 2004, 43, 1389.
[14] Mitsukami, Y.; Donovan, M. S.; Lowe, A. B.; McCormick, C. L. Macromolecules 2001, 34, 2248-2256.
[15] Wooley, K. L. J. Polym. Sci., Part A: Polym. Chem. 2000, 38, 1397-1401.
[16] Thurmond, K. B.; Kowalewski, T.; Wooley, K. L. J. Am. Chem. Soc. 1997, 119, 6656-6665.
[17] Huang, H.; Remsen, E. E.; Wooley, K. L. Chem. Commun. 1998, 1415-1416.
[18] Huang, H.; Remsen, E. E.; Kowalewski, T.; Wooley, K. L. J. Am. Chem. Soc. 1999, 121, 3805-3806.
[19] Ma, Q.; Wooley, K. L. J. Polym. Sci., Part A: Polym. Chem. 2000, 38, 4805-4820.
[20] Becker, M. L.; Remsen, E. E.; Wooley. K. L. J. Polym. Sci., Part A: Polym. Chem. 2001, 39, 4152-4166.
[21] Ma, Q.; Remsen, E. E.; Kowalewski, T.; Wooley, K. L. J. Am. Chem. Soc. 2001, 123, 4627-4628.
[22] Sanji, T.; Nakatsuka, Y.; Kitayama, F.; Sakurai, H. Chem. Commun. 1999, 2201-2202.
[23] Sanji, T.; Nakatsuka, Y.; Ohnishi, S.; Sakurai, H. Macromolecules 2000, 33, 8524-8526.
[24] Underhill, R. S.; Liu, G. Chem. Mater. 2000, 12, 2082-2091.
[25] Fujii, S.; Cai, Y.; Weaver, J. V M.; Armes, S. P. J. Am. Chem. Soc. 2005, 127, 7304-7305.
[26] Butuen, V.; Wang, X.-S.; de Paz Banez, M. V.; Robinson, K. L.; Billingham, N. C.; Armes, S. P. Macromolecules 2000, 33, 1-3.
[27] Liu, S.; Armes, S. P. J. Am. Chem. Soc. 2001, 123, 9910-9911.
[28] Thurmond, K. B.; Kowalewski, T.; Wooley, K. L. J. Am. Chem. Soc. 1996, 118, 7239-7240.
[29] Huang, H.; Kowalewski, T.; Remsen, E. E.; Gertzmann, R.; Wooley, K. L.; J. Am. Chem. Soc. 1997, 119, 11653-11659.
[30] Zhang, Q.; Remsen, E. E.; Wooley, K. L. J. Am. Chem. Soc. 2000, 122, 3642-3651.
[31] Butuen, V; Billingham, N. C.; Armes, S. P. J. Am. Chem. Soc. 1998, 120, 12135-12136.
[32] Butuen, V.; Lowe, A. B.; Billingham, N. C.; Armes, S. P. J. Am. Chem. Soc. 1999, 121, 4288-4289.
[33] Pilon, L. N.; Armes, S. P.; Findlay, P.; Rannard, S. P. Langmuir 2005, 21, 3808-3813.
[34] Liu, S.; Weaver, J. V. M.; Save, M.; Armes, S. P. Langmuir 2002, 18, 8350-8357.
[35] Stewart, S.; Liu, G. J. Chem. Mater. 1999, 11, 1048-1054.
[36] Sanada, F.; Endo, T. Macromol. Chem. Phys. 1999, 200, 2651-2661.
[37] Sanada, F.; Abe, T.; Endo, T. J. Polym. Sci., Part A: Polym. Chem. 1997, 35, 2619-2629.
[38] Mori, H.; Sutoh, K.; Endo, T. Macromolecules 2005, 38, 9055-9065.
[39] Matyjaszweski, K., Ed. Controlled/Living Radical Polymerization; American Chemical Society: Washington D.C., 2003.
[40] Hawker, C. J.; Bosman, A. W.; Harth, E. Chem. Rev. 2001, 101, 3661-3688.
[41] Matyjaszewski, K.; Xia, J. Chem. Rev. 2001, 101, 2921-2990.
[42] Moad, G.; Rizzardo, E.; Thang, S. H. Austr. J. Chem. 2005, 58, 379-410.
[43] Chiefari, J.; Chong, Y. K.; Ercole, F.; Krstina, J.; Jeffery, J.; Le, T. P. T.; Mayadunne, R. T. A.; Meijs, G. F.; Moad, C. L.; Moad, G.; Rizzardo, E.; Thang, S. H. Macromolecules 1998, 31, 5559-5562.
[44] Perrier, S.; Takolpuckdee, P. J. Polym. Sci., Part A. Polym. Chem. 2005, 43, 5347-5393.
[45] Sumerlin, B. S.; Donovan, M. S.; Mitsukami, Y.; Lowe, A. B.; McCormick, C. L. Macromolecules 2001, 34, 6561-6564.
[46] Sumerlin, B. S.; Lowe, A. B.; Thomas, D. B.; McCormick, C. L. Macromolecules 2003, 36, 5982-5987.
[47] Vasilieva, Y. A.; Thomas, D. B.; Scales, C. W.; McCormick, C. L. Macromolecules 2004, 37, 2728-2737.
[48] Donovan, M. S.; Lowe, A. B.; Sanford, T. A.; McCormick, C. L. J. Polym. Sci., Part A: Polym. Chem. 2003, 41, 1262-1281.
[49] Donovan, M. S.; Sumerlin, B. S.; Lowe, A. B.; McCormick, C. L. Macromolecules 2002, 35, 8663-8666.
[50] Donovan, M. S.; Sanford, T. A.; Lowe, A. B.; Sumerlin, B. S.; Mitsukami, Y.; McCormick, C. L. Macromolecules 2002, 35, 4570-4572.
[51] Thomas, D. B.; Sumerlin, B. S.; Lowe, A. B.; McCormick, C. L. Macromolecules 2003, 36, 1436-1439.
[52] Convertine, A. J.; Ayres, N.; Scales, C. W.; Lowe, A. B.; McCormick, C. L.; Biomacromolecules 2004, 5, 1177-1180.
[53] Convertine, A. J.; Lokitz, B. S.; Vasileva, Y.; Myrick, L. J.; Scales, C. W.; Lowe, A. B.; McCormick, C. L. Macromolecules 2006, 5, 1724-1730.
[54] Thomas, T. B.; Convertine, A. J.; Myrick, L. J.; Scales, C. W.; Smith, A. E.; Lowe, A. B.; Vasiliva, Y. A.; Ayres, N.; McCormick, C. L. Macromolecules 2004, 37, 8941-8950.
[55] Camail, M.; Margaillan, A.; Maesano, J. C.; Thuret, S.; Vemet, J. L. Polymer 1998, 39, 3187-3192.
[56] Heskins, M.; Guillet, J. E. J. Macromol. Sci. Chem. 1968, A2, 1441-1455.
[57] Cho, E. C.; Lee, J.; Cho, K. Macromolecules 2003, 36, 9929-9934.
[58] Yusa, S.; Shimada, Y.; Mitsukami, Y.; Yamamoto, T.; Morishima, Y. Macromolecules 2004, 37, 7507-7513.
[59] Liu, B.; Perrier, S. J. Polym. Sci., Part A: Polym. Chem. 2005, 43, 3643-3654.
[60] Greenwood, R. Adv. Colloid Interface Sci. 2003, 106, 55-81.
[61] Sumerlin, B. S.; Lowe, A. B.; Thomas, D. B.; Convertine, A. J.; Donovan, M. S.; McCormick, C. L. J. Polym. Sci., Part A: Polym. Chem. 2004, 42, 1724-1734.
[62] Kabanov, A. V.; Alakhov, V. Yu. Crit. Rev. Ther. Drug Carrier Syst. 2002, 19, 1-72.
[63] Otsuka, H.; Nagasaki, Yu.; Kataoka, K. Adv. Drug Delivery Rev. 2003, 55, 403-419.
[64] Bronich, T. K.; Keifer, P. A.; Shlyakhtenko, L. S.; Kabanov, A. V. J. Am. Chem. Soc. 2005, 127, 8236-8237.
[65] Li, Y.; Lokitz, B.; McCormick, C. L. Macromolecules, 2005, 39(1); 81-89.
[66] Allen, C.; Maysinger, D.; Eisenberg, A. Colloids Surf B: Biointerfaces 1999, 16, 3-27.
[67] Cline, D. J.; Redding, S. E.; Brohawn, S. G.; Psathas, J. N.; Schneider, J. P.; Thorpe, C.; Biochemistry, 2004, 43, 15195-15203.
[68] (a) Tsarevsky, N. V.; Matyjaszewski, K. Macromolecules, 2002, 35, 9009-9014. (b) Aliyar, H. A.; Hamilton, P. D.; Ravi, N. Biomacromolecules, 2005, 6, 204-211. (c) Plunkett, K. N.; Berkowski, K. L.; Moore, J. S. Biomacromolecules, 2005, 6, 632-637. (d) Li, Y.; Armes, S. P.; Macromolecules, 2005; 38; 8155-8162.
[69] Aliyar, H. A.; Hamilton, P. D.; Ravi, N. Biomacromolecules, 2005, 6, 204-211.
[70] Grisham, M. B. Free Radical Biology and Medicine, 2004, 36, 1479-1480.

1. A thermally responsive, reversible crosslinked nanostructure comprising:
a multiblock copolymer comprising a first block of a charged polymer, and a second block of a thermally responsive polymer, said multiblock copolymer crosslinked with a polymer having the opposite charge of the first block polymer.

2. The nanostructure of claim 1 which is a vesicle or micelle.

3. The nanostructure of claim 1 further comprising a bioactive compound.

4. The nanostructure of claim 1 wherein the thermally responsive polymer is poly(N-isopropylacrylamide) (NIPAM).

5. The nanostructure of claim 1 wherein the thermally responsive polymer is poly(2-[(N-morpholino)ethyl]methacrylate), poly(N-acryloylpyrrolidine), poly(N-acryloylpiperidine), or poly(N-n-propylacrylamide).

6. The nanostructure of claim 1 wherein the charged polymer is an anionic polymer.

7. The nanostructure of claim 6 wherein the anionic polymer is sulfonated or carboxylated polystyrene, sulfonated or carboxylated polyacrylamide, sulfonated or carboxylated polymethacrylamide, sulfonated or carboxylated polyacrylate, or sulfonated or carboxylated polymethacrylate.

8. The nanostructure of claim 6 wherein the anionic polymer is poly(sodium 2-acrylamido-2-methylpropanesulfonate) (PAMPS)

9. The nanostructure of claim 6 wherein the anionic polymer is a poly(acryloylamino acid).

10. The nanostructure of claim 9 wherein the anionic polymer is poly(N-acryloyl alanine) (AAL) or poly(N-acryloyl valine) (AVAL).

11. The nanostructure of claim 1 wherein the charged polymer is a cationic polymer.

12. The nanostructure of claim 11 wherein the cationic polymer is a protonated or quaternized polyvinylbenzylamine, polyaminomethacrylamide, polyaminoacrylamide, polyaminomethacrylate, polyaminoacrylate, or polyvinylpyrridines.

13. The nanostructure of claim 11 wherein the cationic polymer is poly[(ar-vinyl benzyl) ammonium chloride] (PVBTAC) or poly(N-(3-aminopropyl)methacrylamide hydrochloride) (AMPA).

14. The nanostructure of claim 1 wherein the multiblock copolymer is poly(AMPA)-block-(NIPAM).

15. The nanostructure of claim 1 wherein the multiblock copolymer is poly(AAL)-block-poly(NIPAM) or poly(AVAL)-block-poly(NIPAM).

16. The nanostructure of claim 1 wherein the multiblock copolymer is poly(PAMPS)-block-poly(NIPAM).

17. The nanostructure of claim 1 wherein the multiblock copolymer further comprises a third block of a nonionic, hydrophilic polymer.

18. The nanostructure of claim 17 wherein the nonionic, hydrophilic polymer is a polyacrylamide, polymethacrylamide, polyacrylate, polymethacrylate, polyacryloyl morpholine, polyvinyl pyrrolidone, glycopolymer, polyethylene glycol methacrylate, polyalkylene oxide, or polyvinyl alcohol.

19. The nanostructure of claim 17 wherein the third block is poly(N,N,-dimethyl acrylamide) (DMA) or poly(ethylene oxide) (PEO).

20. The nanostructure of claim 18 wherein the multiblock copolymer is poly(DMA)-block-poly(AAL)-block-poly(NIPAM) or poly(DMA)-block-poly(AVAL)-block-poly(NIPAM).

21. The nanostructure of claim 18 wherein the multiblock copolymer is poly(DMA)-block-poly(AAL)-block-poly(NIPAM)-block-poly(AAL)-block-poly(DMA) or poly(DMA)-block-poly(AVAL)-block-poly(NIPAM)-block-poly(AVAL)-block-poly(DMA).

22. A method of forming thermally responsive, reversible crosslinked nanostructure comprising:
synthesizing in solution via RAFT polymerization a multiblock copolymer comprising a first block of a charged polymer and a second block of a thermally responsive polymer,
raising the solution temperature of the synthesized multiblock copolymer to above the lower critical solution temperature of the second block, and
contacting said multiblock copolymer with a polymer having the opposite charge of the first block polymer to form a crosslinked nanostructure.

23. The method of claim 22 further comprising adjusting the pH of the solution to above 5.

24. The method of claim 22 wherein the nanostructure is a micelle or vesicle.

25. The method of claim 22 further comprising after raising the solution temperature, adding a bioactive agent to the multiblock copolymer.

26. A thermally responsive, reversible crosslinked nanostructure comprising:
a multiblock copolymer comprising a first block of a copolymer of N-acryloxysuccinimide (NAS) or N-methacryloxysuccinimide (NMS) and a nonionic, hydrophilic monomer, and a second block of a thermally responsive polymer, said multiblock copolymer crosslinked with cystamine.

27. The nanostructure of claim 26 which is a vesicle or micelle.

28. The nanostructure of claim 26 further comprising a bioactive compound.

29. The nanostructure of claim 26 wherein the thermally responsive polymer is poly(N-isopropylacrylamide) (NIPAM).

30. The nanostructure of claim 26 wherein the thermally responsive polymer is poly(2-[(N-morpholino)ethyl]methacrylate), poly(N-acryloylpyrrolidine), poly(N-acryloylpiperidine), or poly(N-n-propylacrylamide).

31. The nanostructure of claim 26 wherein the first block is poly[(DMA)-stat-(NAS)].

32. The nanostructure of claim 26 wherein the multiblock copolymer further comprises a third block of a nonionic, hydrophilic polymer.

33. The nanostructure of claim 32 wherein the nonionic, hydrophilic polymer is a polyacrylamide, polymethacrylamide, polyacrylate, polymethacrylate, polyacryloyl morpholine, polyvinyl pyrrolidone, glycopolymer, polyethylene glycol methacrylate, polyalkylene oxide, polyvinyl alcohol.

34. The nanostructure of claim 32 wherein the third block is poly(DMA) or (PEO).

35. The nanostructure of claim 32 wherein the multiblock copolymer comprises (PEO)-block-poly[(DMA)-stat-(NAS)]-block-poly(NIPAM).

36. The nanostructure of claim 26 which is reversible by a cleaving agent.

37. The nanostructure of claim 36 wherein the cleaving agent is tris(2-carboxyethyl)phosphine or dithiothreitol.

38. A method for the controlled polymerization of an unprotected amino acid-based monomer directly in water, the method comprising:
RAFT polymerizing an unprotected acryloyl or methacryloyl amino acid monomer in water with a chain transfer agent and a free radical initiator and forming an acryloyl or methacryloyl amino acid polymer.

39. The method of claim 38 where the unprotected amino acid is alanine or valine.

40. The method of claim 38 where the chain transfer agent is 4-cyanopentanoic acid dithiobenzoate (CTP) or 2-ethylsulfanylthiocarbonylsulfonyl-2-methyl-propionic acid (EMP) and the free radical initiator is 4,4′-azobis(4-cyanopentanoic acid).
